(19) United States
(12) Patent Application Publication
(10) Pub. No.: US 2025/0121152 A1
(43) Pub. Date: Apr. 17, 2025

(54) NASAL TUBE DEVICES FOR SECURING A NASAL TUBE AND ONE OR MORE OTHER TUBES TO A NOSE OF A PATIENT

(71) Applicant: APPLIED MEDICAL TECHNOLOGY, INC., Brecksville, OH (US)

(72) Inventors: George PICHA, III, Loomis, CA (US); George J. PICHA, Brecksville, OH (US); Steven Alfred SOEDER, North Royalton, OH (US); Benjamin DALESSANDRO, Brecksville, OH (US); Grant Wesley PHILLIPS, Richfield, OH (US); Derek M. WILLIAMS, Cuyahoga Falls, OH (US)

(21) Appl. No.: 18/809,819

(22) Filed: Aug. 20, 2024

Related U.S. Application Data
(60) Provisional application No. 63/589,976, filed on Oct. 12, 2023.

(51) Int. Cl. A61M 16/06; A61J 15/00; A61M 16/08

(52) U.S. Cl. CPC A61M 16/0688; A61J 15/0003; A61J 15/0061; A61M 16/0672; A61M 16/085

(57) ABSTRACT

A nasal tube device for securing a nasal tube and one or more other tubes to a nose of a patient is disclosed. The nasal tube device comprises: (a) a nasal tube; (b) a flange extending radially outwardly from a proximal-flange portion of the nasal tube; (c) an adhesive tape being attached to the flange at a base portion of the adhesive tape; and (d) a clip for securing one or more other tubes to the nasal tube within a lumen of the nasal tube at the proximal-clip portion of the nasal tube, the clip being reversibly closeable about the proximal-clip portion of the nasal tube to reversibly compress the proximal-clip portion of the nasal tube about the one or more other tubes when closed. The flange is highly compliant and provides a barrier to moisture and heat. The nasal tube has an elliptical tubular cross section that is noncircular.

### CROSS-REFERENCE

This application claims the benefit of U.S. Provisional Application No. 63/589,976, filed Oct. 12, 2023, which is hereby incorporated by reference.

### FIELD OF THE INVENTION

The present invention relates generally to nasal tube devices, and more particularly to nasal tube devices for securing a nasal tube and one or more other tubes to a nose of a patient.

### BACKGROUND OF THE INVENTION

A nasal tube is a flexible tube that is inserted into a nostril of a patient and advanced through the nasal cavity of the patient. Examples of nasal tubes include nasopharyngeal airways, also termed NPAs, which can be used for non-definitive airway management, nasotracheal tubes, also termed NT tubes, which can be used to provide a secure airway for patients who require mechanical ventilation, nasogastric tubes, also termed NG tubes, which can be used to deliver liquids, food, and medications to the stomach or to remove substances from the stomach, and nasojejunal tubes, also termed NJ tubes, which can be used to deliver nutrients to the small intestine.

A nasal tube may terminate prior to the patient's nasopharynx or may pass beyond the nasopharynx. For examples, nasopharyngeal airways typically terminate in the patient's oropharynx, proximal to the epiglottis.

Nasopharyngeal airways are commonly used in monitored anesthesia care procedures, also termed MAC procedures. Nasopharyngeal airways are used to bypass the tongue and provide a patent lumen through the natural airway, avoiding obstruction of the airway caused by the loss of muscle tone resulting from sedation. During MAC procedures, the standard of care is to use capnography to monitor the patient's respiratory function. In addition to capnography, many patients require supplemental oxygenation to maintain target blood oxygen levels. A divided nasal cannula is commonly used to deliver oxygen and sample respirations for capnography. However, in many patients, superficial oxygen delivery and respiratory sampling may be insufficient to achieve target blood-oxygen levels or obtain a quality capnography signal. In such cases, it is common practice to cut the nose piece from the nasal sampling cannula and insert the resulting cut oxygen supply and respiratory sampling tubes down the lumen of a nasopharyngeal airway, to access the oropharynx for oxygen delivery and respiratory sampling. Delivering oxygen through a nasopharyngeal airway results in less dilution with ambient air than does superficial delivery, which leads to higher oxygen concentrations in the oropharynx and improved oxygen absorption. Sampling exhaled gas directly from the oropharynx provides a more accurate and reliable capnography signal than does superficial delivery, while avoiding dilution of the sample with ambient air. Maintaining two separate tubes for oxygen delivery and respiratory sampling allows the insertion depth of the capnography pickup point to be adjusted independent of the oxygen delivery point, which can be helpful countering the effects of oxygen washout of the respiratory signal. Moreover, having both tubes and the nasopharyngeal airway placed in a single nostril helps to minimize the equipment footprint without impeding upon access through the opposite nostril or the mouth.

However, absent a means to secure the oxygen supply line and respiratory sampling line, the lines can easily become displaced. Displacement of the oxygen supply line can lead to reduced oxygen concentrations and can cause issues with the capnography signal through respiratory sample washout. Displacement of the capnography sampling line can lead to reduced capnography signal quality or loss of signal. Therefore, it is important to secure the lines after placement.

In patients having difficult or critical airways, it is important to secure the nasopharyngeal airway itself, to avoid displacement of the nasopharyngeal airway. This is typically accomplished using an adhesive tape to secure the airway externally to the skin of the patient's nose or face. Unfortunately, this is not easily done with currently existing nasopharyngeal airways.

For example, currently existing nasopharyngeal airways generally lack a suitable surface space for adhesive tape attachment, requiring creative solutions to attach an adhesive tape to the nasopharyngeal airway, such as piercing the tube of a nasopharyngeal airway with two safety pins to provide anchor points for the adhesive tape (Bajaj et al., The Journal of Laryngology & Otology, vol. 122, pages 733-734 (2008)).

Moreover, even when nasopharyngeal airways provide suitable surface space for adhesive attachment, the plurality of adhesive tapes and nasopharyngeal airways on the market having varying chemical makeups means ad-hoc tape selection rarely achieves optimal adhesive securement and can make it difficult or impossible to achieve a good bond between an adhesive tape and a nasopharyngeal airway. This is because interfacial adhesive strength is primarily determined by the intermolecular interactions at the adhesive-substrate interfaces. Contamination of the nasopharyngeal airway by mucosal secretions during nasopharyngeal airway introduction, prior to tape application, also may lead to poor interfacial strength. Hydrolysis also may lead to loss of interfacial strength as the adhesive is exposed to nasal secretions and to warm, moist air being exhaled through the nose. Accordingly, poor adhesive bonding between the adhesive tape and the nasopharyngeal airway may result in poor securement and/or rapid loss of securement after a seemingly secure initial placement once the adhesive tape is exposed to the warm, humid environment of the nostrils in spontaneously breathing patients.

Additionally, securing a nasopharyngeal airway with an adhesive tape that has not been pre-selected for safe use on the face can result in serious skin injuries. Also, the lack of dedicated tape attachment points on existing nasopharyngeal airways can require routing an adhesive tape in a manner that creates pressure points, which may result in a pressure injury.

To keep the nasopharyngeal airway in place, existing nasopharyngeal airways generally rely on a friction fit. The nasal tube will generally feature a circular tubular cross section, which must be compressed to squeeze through the narrow stricture between the septum and inferior turbinate. This compression of a round tube having a circular tubular cross section results in significant pressure at the tube-tissue interface, which creates the force that keeps the nasopharyngeal airway in place. However, the increased pressure and resulting forces exerted on the nasal tissues creates a high risk of injury during insertion and advancement of the nasopharyngeal airway. Since these nasal tissues are highly vascularized, injury to these tissues often results in bleeding, which can be highly disruptive to a surgical procedure. As blood drains to the pharynx, it can trigger coughing and gagging fits that can interrupt the procedure and compromise ventilation of the patient. Failure to obtain hemostasis quickly can result in termination of the procedure, resulting in delay of care. Indeed bleeding in patients caused by injury during insertion and advancement of nasopharyngeal airways is of such concern among anesthesiologists that some anesthesiologists instead use endotracheal tubes and deeper anesthesia on patients, resulting in other disadvantages to these patients.

U.S. Pat. No. 10,426,941 discloses a nasal tube device intended to address these issues. The nasal tube has a proximal portion, a distal portion, and one or more lumens. A securing device may be attached to the proximal portion of the nasal tube and may be configured to attach a flexible line of a retention system to the nasal tube. The securing device may also be configured to selectively compress the proximal portion of the nasal tube. In certain embodiments, one or more medical tubes are disposed in the lumen of the nasal tube and may be selectively secured in the lumen with the securing device. The nasal tube device may also have a retention flange for attaching the flexible line of the retention system to the nasal tube.

Unfortunately, the process of placing and securing the nasal tube device of U.S. Pat. No. 10,426,941 to patients is time consuming and invasive. It involves inserting a flexible line through one nostril, inserting a retrieval probe through the other nostril, connecting the flexible line to the retrieval probe within the nasopharynx, pulling the flexible line around the vomer bone and out the second nostril while simultaneously advancing the nasal tube through the first nostril, and then tying the two ends of the flexible line together to secure the nasal tube around the vomer bone. Moreover, once the flexible line has been secured, removal of the nasal tube device necessitates cutting the flexible line, which requires the use of a tool, such as scissors. This can increase risk in critical situations like cardiac arrest, where a swift transition to mechanical ventilation is required to provide emergency care.

Accordingly, a need exists for improved nasal tube devices for securing a nasal tube and one or more other tubes to a nose of a patient. A need also exists for such improved devices that decrease the risk of trauma during insertion of the nasal tubes in nostrils of patients.

### BRIEF SUMMARY OF THE INVENTION

A nasal tube device for securing a nasal tube and one or more other tubes to a nose of a patient is disclosed.

The nasal tube device comprises:
(a) a nasal tube comprising, sequentially, a proximal end, a proximal-clip portion, a proximal-flange portion, a distal portion, and a distal end, the proximal end of the nasal tube having a proximal opening, the distal end of the nasal tube having a distal opening, and the nasal tube having a nasal tube lumen extending from the proximal opening of the nasal tube to the distal opening of the nasal tube;
(b) a flange having a proximal face and a distal face, the flange extending radially outwardly from the proximal-flange portion of the nasal tube, the nasal tube extending through the flange;
(c) an adhesive tape comprising a base portion and one or more strip portions extending from the base portion, the base portion of the adhesive tape having a proximal side, a distal side, and a hole therethrough, each of the one or more strip portions having a proximal side and a distal side, the distal side of each of the one or more strip portions each having an adhesive surface, the nasal tube extending through the hole of the base portion of the adhesive tape, the adhesive tape being attached to the flange at the base portion of the adhesive tape; and
(d) a clip for securing one or more other tubes to the nasal tube within the lumen of the nasal tube at the proximal-clip portion of the nasal tube, the clip being reversibly closeable about the proximal-clip portion of the nasal tube to reversibly compress the proximal-clip portion of the nasal tube about the one or more other tubes when closed.

The flange is highly compliant based on being thin and flexible and provides a barrier to moisture and heat based on its composition.

The nasal tube has an elliptical tubular cross section that is noncircular.

In some embodiments, the nasal tube comprises a nasopharyngeal airway, a nasotracheal tubes, a nasogastric tube, or a nasojejunal tube.

In some embodiments, the flange is formed integrally to the nasal tube at the proximal-flange portion of the nasal tube.

In some embodiments, the flange is formed separately from the nasal tube, the flange has a hole therethrough, and the nasal tube extends through the hole of the flange.

In some embodiments, the flange has a thickness of 0.50 to 5.00 mm, 0.60 to 2.50 mm, or 0.75 to 1.00 mm.

In some embodiments, the flange is made from a material having (i) a tensile strength of 2.0 to 20 MPa, a tensile modulus of 0.5 to 10 MPa, and a hardness of 50 Shore-00 to 90 Shore A, (ii) a tensile strength of 5 to 15 MPa, a tensile modulus of 1 to 5 MPa, and a hardness of 30 to 70 Shore A, or (iii) a tensile strength of 8 to 10 MPa, a tensile modulus of 2 to 3 MPa, and a hardness of 40 to 60 Shore A.

In some embodiments, the flange is made from one or more of silicone rubber elastomer, natural latex rubber, thermoplastic elastomer, styrene block copolymer, thermoplastic polyolefin, thermoplastic polyurethane, thermoplastic vulcanizate, ethylene-propylene-diene-monomer, copolymer derivatives of ethylene-propylene-diene-monomer, styrene-butadiene-rubber, copolymer derivatives of styrene-butadiene-rubber, flexible non-elastomeric polymer, or plasticized polyvinyl chloride.

In some embodiments, the one or more strip portions of the adhesive tape comprise two to four strip portions extending from the base portion of the adhesive tape.

In some embodiments, the adhesive tape is attached to the flange based on the distal side of base portion of the adhesive tape being adhered to the proximal face of the flange.

In some embodiments, the adhesive tape is attached to the flange based on the proximal side of base portion of the adhesive tape being adhered to the distal face of the flange.

In some embodiments, the nasal tube device further comprises an adhesive liner, the adhesive liner covering the adhesive surfaces of the distal sides of the one or more strip portions of the adhesive tape.

In some embodiments, the clip and the proximal-clip portion of the nasal tube comprise structures that provide a complementary fit between the clip and the proximal-clip portion of the nasal tube when the clip is closed about the proximal-clip portion of the nasal tube.

In some embodiments, one or more of the clip or the proximal-clip portion of the nasal tube comprise one or more protrusions to reversibly compress the proximal-clip portion of the nasal tube about the one or more other tubes when the clip is closed about the proximal-clip portion of the nasal tube.

In some embodiments, the nasal tube has an elliptical tubular cross section that is noncircular based on the elliptical tubular cross section having a major axis length and an interfocal length, and the interfocal length being 0.50 to 0.99 times the major axis length.

A system for securing a nasal tube and one or more other tubes to a nose of a patient also is disclosed. The system comprises the nasal tube device comprising (a) a nasal tube, (b) a flange, (c) an adhesive tape, and (d) a clip as described above. The system also comprises one or more other tubes. The one or more other tubes are partially disposed in the nasal tube lumen of the nasal tube through the proximal opening of the nasal tube. The clip is reversibly closed about the proximal-clip portion of the nasal tube, thereby reversibly compressing the proximal-clip portion of the nasal tube about the one or more other tubes.

In some embodiments, the nasal tube comprises a nasopharyngeal airway, and the one or more other tubes comprise a tube for oxygen delivery and a tube for carbon dioxide monitoring.

Another nasal tube device for securing a nasal tube and one or more other tubes to a nose of a patient also is disclosed.

The nasal tube device comprises:
(a) a nasal tube comprising, sequentially, a proximal end, a proximal-clip portion, a distal portion, and a distal end, the proximal end of the nasal tube having a proximal opening, the distal end of the nasal tube having a distal opening, and the nasal tube having a nasal tube lumen extending from the proximal opening of the nasal tube to the distal opening of the nasal tube;
(b) a bolster comprising a proximal end, a bolster body, and a distal end, the proximal end of the bolster having a proximal opening, the distal end of the bolster having a distal opening, the bolster having a bolster lumen extending from the proximal opening of the bolster to the distal opening of the bolster, the bolster being formed separately from the nasal tube, the nasal tube extending through the bolster lumen;
(c) a flange having a proximal face and a distal face, the flange extending radially outwardly from the nasal tube, the flange having a hole between the proximal face of the flange and the distal face of the flange, the flange being formed separately from the nasal tube, the nasal tube extending through the hole of the flange, the flange being distal to the bolster on the nasal tube;
(d) an adhesive tape comprising a base portion and one or more strip portions extending from the base portion, the base portion of the adhesive tape having a proximal side, a distal side, and a hole therethrough, each of the one or more strip portions having a proximal side and a distal side, the distal side of each of the one or more strip portions each having an adhesive surface, the nasal tube extending through the hole of the base portion of the adhesive tape, the adhesive tape being attached to the flange at the base portion of the adhesive tape; and
(e) a clip for securing one or more other tubes to the nasal tube within the lumen of the nasal tube at the proximal-clip portion of the nasal tube, the clip being reversibly closeable about the bolster to reversibly compress the proximal-clip portion of the nasal tube about the one or more other tubes when closed.

The flange is highly compliant based on being thin and flexible and provides a barrier to moisture and heat based on its composition.

The nasal tube has an elliptical tubular cross section that is noncircular.

In some embodiments, the nasal tube comprises a nasopharyngeal airway, a nasotracheal tubes, a nasogastric tube, or a nasojejunal tube.

In some embodiments, the flange has a thickness of 0.50 to 5.00 mm, 0.60 to 2.50 mm, or 0.75 to 1.00 mm.

In some embodiments, the flange is made from a material having (i) a tensile strength of 2.0 to 20 MPa, a tensile modulus of 0.5 to 10 MPa, and a hardness of 50 Shore-00 to 90 Shore A, (ii) a tensile strength of 5 to 15 MPa, a tensile modulus of 1 to 5 MPa, and a hardness of 30 to 70 Shore A, or (iii) a tensile strength of 8 to 10 MPa, a tensile modulus of 2 to 3 MPa, and a hardness of 40 to 60 Shore A.

In some embodiments, the flange is made from one or more of silicone rubber elastomer, natural latex rubber, thermoplastic elastomers, styrene block copolymers, thermoplastic polyolefins, thermoplastic polyurethanes, thermoplastic vulcanizates, ethylene-propylene-diene-monomer, copolymer derivatives of ethylene-propylene-diene-monomer, styrene-butadiene-rubber, copolymer derivatives of styrene-butadiene-rubber, flexible non-elastomeric polymers, or plasticized PVC.

In some embodiments, the flange extends from the bolster at the distal end of the bolster. In some of these embodiments, the flange is integral to the bolster. In some of these embodiments, the flange is formed separately from the bolster and is attached to the bolster.

In some embodiments, the bolster is slidable along the nasal tube when the clip is not reversibly closed about the bolster, and the flange is slidable along the nasal tube. In some of these embodiments, the nasal tube further comprises a rim extending radially outwardly from the nasal tube at the proximal end of the nasal tube, the rim having an outer diameter greater than an inner diameter of the bolster lumen and an inner diameter of the hole of the base portion of the adhesive tape.

In some embodiments, the one or more strip portions of the adhesive tape comprise two to four strip portions extending from the base portion of the adhesive tape.

In some embodiments, the adhesive tape is attached to the flange based on the distal side of base portion of the adhesive tape being adhered to the proximal face of the flange.

In some embodiments, the adhesive tape is attached to the flange based on the proximal side of base portion of the adhesive tape being adhered to the distal face of the flange.

In some embodiments, the nasal tube device further comprises an adhesive liner, the adhesive liner covering the adhesive surfaces of the distal sides of the one or more strip portions of the adhesive tape.

In some embodiments, the clip and the bolster comprise structures that provide a complementary fit between the clip and the bolster of the nasal tube when the clip is closed about the bolster.

In some embodiments, one or more of the clip or the bolster comprise one or more protrusions to reversibly compress the proximal-clip portion of the nasal tube about the one or more other tubes when the clip is closed about the bolster.

In some embodiments, the nasal tube has an elliptical tubular cross section that is noncircular based on the elliptical tubular cross section having a major axis length and an interfocal length, and the interfocal length being 0.50 to 0.99 times the major axis length.

Another system for securing a nasal tube and one or more other tubes to a nose of a patient also is disclosed. The system comprises the nasal tube device comprising (a) a nasal tube, (b) a bolster, (c) a flange, (d) an adhesive tape, and (e) a clip, as described above. The system also comprises one or more other tubes. The one or more other tubes are partially disposed in the nasal tube lumen of the nasal tube through the proximal opening of the nasal tube. The clip is reversibly closed about the bolster, thereby reversibly compressing the proximal-clip portion of the nasal tube about the one or more other tubes.

In some embodiments, the nasal tube comprises a nasopharyngeal airway, and the one or more other tubes comprise a tube for oxygen delivery and a tube for carbon dioxide monitoring.

### BRIEF DESCRIPTION OF THE DRAWINGS

These and other features, aspects, and advantages of the present disclosure are better understood when the following detailed description is read with reference to the accompanying drawings.

FIG. 1 is a perspective view of an embodiment of a system for securing a nasal tube and one or more other tubes to a nose of a patient comprising a nasal tube device comprising (a) a nasal tube, (b) a flange, (c) an adhesive tape, and (d) a clip, as disclosed herein, and one or more other tubes corresponding to a tube for oxygen delivery and a tube for carbon dioxide monitoring. As shown, the adhesive tape is attached to the flange based on the distal side of base portion of the adhesive tape being adhered to the proximal face of the flange. Also, the clip is closed. In addition, the nasal tube device further comprises an adhesive liner disposed along major portions of three strip portions of the adhesive tape.

FIG. 2 is a first perspective view of the nasal tube device and portions of the one or more other tubes of the system of FIG. 1.

FIG. 3 is a second perspective view of the nasal tube device and portions of the one or more other tubes of the system of FIG. 1.

FIG. 4 is a front view of the nasal tube device and portions of the one or more other tubes of the system of FIG. 1.

FIG. 5 is a back view of the nasal tube device and portions of the one or more other tubes of the system of FIG. 1.

FIG. 6 is a first side view of the nasal tube device and portions of the one or more other tubes of the system of FIG. 1.

FIG. 7 is a second side view of the nasal tube device and portions of the one or more other tubes of the system of FIG. 1.

FIG. 8 is a top view of the nasal tube device and portions of the one or more other tubes of the system of FIG. 1.

FIG. 9 is a bottom view of the nasal tube device and portions of the one or more other tubes of the system of FIG. 1.

FIG. 10 is a sectional view of the nasal tube device and portions of the one or more other tubes of the system of FIG. 1, viewed along the cutting plane shown in FIG. 4.

FIG. 11 is a perspective view of an embodiment of a nasal tube device comprising (a) a nasal tube, (b) a flange, (c) an adhesive tape, and (d) a clip, as disclosed herein. As shown, the adhesive tape is attached to the flange based on the distal side of base portion of the adhesive tape being adhered to the proximal face of the flange. Also, the clip is open. In addition, the nasal tube device further comprises an adhesive liner disposed along major portions of three strip portions of the adhesive tape.

FIG. 12 is a first perspective view of the nasal tube and the flange of the nasal tube device of FIG. 11.

FIG. 13 is a second perspective view of the nasal tube and the flange of FIG. 12.

FIG. 14 is a front view of the nasal tube and the flange of FIG. 12.

FIG. 15 is a back view of the nasal tube and the flange of FIG. 12.

FIG. 16 is a first side view of the nasal tube and the flange of FIG. 12.

FIG. 17 is a second side view of the nasal tube and the flange of FIG. 12.

FIG. 18 is a top view of the nasal tube and the flange of FIG. 12.

FIG. 19 is a bottom view of the nasal tube and the flange of FIG. 12.

FIG. 20 is a perspective view of the adhesive tape of the nasal tube device of FIG. 11.

FIG. 21 is a front view of the adhesive tape of FIG. 20.

FIG. 22 is a back view of the adhesive tape of FIG. 20.

FIG. 23 is a perspective view of the adhesive tape of the nasal tube device of FIG. 11. An adhesive liner including a first part disposed at the base portion of the adhesive tape and a second part disposed along major portions of three strip portions of the adhesive tape is also shown.

FIG. 24 is a front view of the adhesive tape and adhesive liner of FIG. 23.

FIG. 25 is a back view of the adhesive tape and adhesive liner of FIG. 23.

FIG. 26 is a perspective view of the adhesive tape and the adhesive liner of the nasal tube device of FIG. 11. As shown, the adhesive liner is disposed along major portions of three strip portions of the adhesive tape.

FIG. 27 is a front view of the adhesive tape and the adhesive liner of FIG. 26.

FIG. 28 is a back view of the adhesive tape and the adhesive liner of FIG. 26.

FIG. 29 is a first perspective view of the nasal tube, flange, adhesive tape, and adhesive liner of the nasal tube device of FIG. 11. As shown, the adhesive liner is disposed along major portions of three strip portions of the adhesive tape.

FIG. 30 is a front view of the nasal tube, flange, adhesive tape, and adhesive liner of FIG. 29.

FIG. 31 is a first perspective view of the clip of the nasal tube device of FIG. 11. As shown, the clip is open. Also, the clip includes protrusions to reversibly compress the proximal-clip portion of the nasal tube about the one or more other tubes when the clip is closed about the proximal-clip portion of the nasal tube.

FIG. 32 is a second perspective view of the clip of FIG. 31.

FIG. 33 is a front view of the clip of FIG. 31.

FIG. 34 is a first perspective view of the clip of the nasal tube device of FIG. 11. As shown, the clip is closed. Also, the clip includes protrusions to reversibly compress the proximal-clip portion of the nasal tube about the one or more other tubes when the clip is closed about the proximal-clip portion of the nasal tube.

FIG. 35 is a second perspective view of the clip of FIG. 34.

FIG. 36 is a front view of the clip of FIG. 34.

FIG. 37 shows steps in operation of the nasal tube device comprising (a) a nasal tube, (b) a flange, (c) an adhesive tape, and (d) a clip, as disclosed herein.

FIG. 38 is a perspective view of the nasal tube, flange, adhesive tape, and clip of the nasal tube device of FIG. 11. As shown, the clip is open.

FIG. 39 is a third perspective view of the nasal tube device and portions of the one or more other tubes of the system of FIG. 1. As shown, the clip is open.

FIG. 40 is a side view of the nasal tube device and portions of the one or more other tubes as shown in FIG. 39.

FIG. 41 is a sectional view of the nasal tube device and portions of the one or more other tubes as shown in FIG. 39, viewed along the cutting plane shown in FIG. 40. As shown, the clip includes protrusions to reversibly compress the proximal-clip portion of the nasal tube about the one or more other tubes when the clip is closed about the proximal-clip portion of the nasal tube.

FIG. 42 is a fourth perspective view of the nasal tube device and portions of the one or more other tubes of the system of FIG. 1. As shown, the clip is closed.

FIG. 43 is a side view of the nasal tube device and portions of the one or more other tubes as shown in FIG. 42.

FIG. 44 is a sectional view of the nasal tube device and portions of the one or more other tubes as shown in FIG. 42, viewed along the cutting plane shown in FIG. 43. As shown, the clip includes protrusions to reversibly compress the proximal-clip portion of the nasal tube about the one or more other tubes when the clip is closed about the proximal-clip portion of the nasal tube.

FIG. 45 is an image of a micrograph of a cross section of a nasal tube, flange, and two other tubes of a system for securing a nasal tube and one or more other tubes to a nose of a patient comprising the nasal tube device comprising (a) a nasal tube, (b) a flange, (c) an adhesive tape, and (d) a clip, as disclosed herein. The adhesive tape and the clip of the nasal tube device are not shown.

FIG. 46 is an image of a micrograph of a cross section of the nasal tube, flange, clip, and two other tubes of a system for securing a nasal tube and one or more other tubes to a nose of a patient comprising the nasal tube device comprising (a) a nasal tube, (b) a flange, (c) an adhesive tape, and (d) a clip, as disclosed herein. The adhesive tape of the nasal tube device is not shown. As shown, the clip is open. Also, the clip and the proximal-clip portion of the nasal tube include protrusions to reversibly compress the proximal-clip portion of the nasal tube about the one or more other tubes when the clip is closed about the proximal-clip portion of the nasal tube.

FIG. 47 is an image of a micrograph of a cross section of the nasal tube, flange, clip, and two other tubes of a system for securing a nasal tube and one or more other tubes shown in FIG. 46. As shown, the clip is closed.

FIG. 48 is a first perspective view of another clip of the nasal tube device comprising (a) a nasal tube, (b) a flange, (c) an adhesive tape, and (d) a clip, as disclosed herein. As shown, the clip is open. Also, the clip does not include protrusions to reversibly compress the proximal-clip portion of the nasal tube about the one or more other tubes when the clip is closed about the proximal-clip portion of the nasal tube.

FIG. 49 is a second perspective view of the clip of FIG. 48.

FIG. 50 is a front view of the clip of FIG. 48.

FIG. 51 is a first perspective view of the clip of FIG. 48, in which the clip is closed.

FIG. 52 is a second perspective view of the clip of FIG. 51.

FIG. 53 is a front view of the clip of FIG. 51.

FIG. 54 is an image of a micrograph of a cross section of a nasal tube, flange, clip, and two other tubes of a system for securing a nasal tube and one or more other tubes to a nose of a patient comprising the nasal tube device comprising (a) a nasal tube, (b) a flange, (c) an adhesive tape, and (d) a clip, as disclosed herein. The adhesive tape of the nasal tube device is not shown. As shown, the clip is closed. Also, the proximal-clip portion of the nasal tube includes a protrusion to reversibly compress the proximal-clip portion of the nasal tube about the one or more other tubes when the clip is closed about the proximal-clip portion of the nasal tube. The clip does not include such a protrusion.

FIG. 55 is an image of a narrow passage between a septum and an inferior turbinate of a human nose.

FIG. 56 is a perspective view of an embodiment of a system for securing a nasal tube and one or more other tubes to a nose of a patient comprising a nasal tube device comprising (a) a nasal tube, (b) a bolster, (c) a flange, (d) an adhesive tape, and (e) a clip, as disclosed herein, and one or more other tubes corresponding to a tube for oxygen delivery and a tube for carbon dioxide monitoring. The view shows the nasal tube device and portions of the one or more tubes. As shown, the clip is open. Also, the nasal tube device further comprises an adhesive liner disposed along major portions of three strip portions of the adhesive tape.

FIG. 57 is a side view of the nasal tube device and portions of the one or more tubes of FIG. 56.

FIG. 58 is a perspective view of the nasal tube and bolster of the nasal tube device of FIG. 56.

FIG. 59 is a perspective view of the nasal tube of the nasal tube device of FIG. 56.

FIG. 60 is a side view of the nasal tube of the nasal tube device of FIG. 56.

FIG. 61 is a first perspective view of the bolster of the nasal tube device of FIG. 56.

FIG. 62 is a second perspective view of the bolster of the nasal tube device of FIG. 56.

FIG. 63 is a side view of the bolster of the nasal tube device of FIG. 56.

FIG. 64 is a first perspective view of the clip of the nasal tube device of FIG. 56. As shown, the clip is open. Also, the clip includes protrusions to reversibly compress the proximal-clip portion of the nasal tube about the one or more other tubes when the clip is closed about the proximal-clip portion of the nasal tube.

FIG. 65 is a second perspective view of the clip of FIG. 64.

FIG. 66 is a sectional view of the nasal tube device and portions of the one or more tubes of FIG. 56, viewed along the cutting plane shown in FIG. 57.

FIG. 67 is a perspective view of an embodiment of a system for securing a nasal tube and one or more other tubes to a nose of a patient comprising a nasal tube device comprising (a) a nasal tube, (b) a bolster, (c) a flange, (d) an adhesive tape, and (e) a clip, as disclosed herein, and one or more other tubes corresponding to a tube for oxygen delivery and a tube for carbon dioxide monitoring. The view shows the nasal tube device and portions of the one or more tubes. As shown, the clip is closed. Also, the nasal tube device further comprises an adhesive liner disposed along major portions of three strip portions of the adhesive tape.

FIG. 68 is a side view of the nasal tube device and portions of the one or more tubes of FIG. 67.

FIG. 69 is a sectional view of the nasal tube device and portions of the one or more tubes of FIG. 67, viewed along the cutting plane shown in FIG. 68.

FIG. 70 is a perspective view of a distal end of a nasal tube as disclosed herein including an axisymmetric tapered tip at the distal end of the nasal tube. As shown, the axisymmetric tapered tip has a perforate distal terminus and ventilation holes.

FIG. 71 is a perspective view of an embodiment of a nasal tube device comprising (a) a nasal tube, (b) a flange, (c) an adhesive tape, and (d) a clip, as disclosed herein. As shown, the adhesive tape is attached to the flange based on the proximal side of base portion of the adhesive tape being adhered to the distal face of the flange. Also, the clip is open.

FIG. 72 is a second perspective view of the nasal tube device of FIG. 71.

### DETAILED DESCRIPTION

To address the need for improved nasal tube devices for securing a nasal tube and one or more other tubes to a nose of a patient, we have developed our nasal tube device comprising (a) a nasal tube, (b) a flange, (c) an adhesive tape, and (d) a clip, as described above, and our nasal tube device comprising (a) a nasal tube, (b) a bolster, (c) a flange, (d) an adhesive tape, and (c) a clip, also as described above. In our nasal tube devices, the flange is highly compliant based on being thin and flexible and provides a barrier to moisture and heat based on its composition, and the nasal tube has an elliptical tubular cross section that is noncircular.

Without wishing to be bound by theory, it is believed that our nasal tube devices as disclosed herein improve upon previous adhesive-tape-based approaches for securing nasal tubes to patients by incorporating an adhesive tape that is pre-attached to the flange and that is pre-selected for material compatibility and skin safety. The flange provides a suitable surface space for adhesive tape attachment. Ad-hoc tape selection and poor adhesive bonding between the adhesive tape and the nasal tube are avoided since the process of adhesive selection is taken out of the hands of the end user. The adhesive tape can be pre-selected for safe use on the face, avoiding serious skin injuries. The flange can be provided with sufficient dedicated tape attachment points to decrease the risk of creating pressure points.

During development of our nasal tube devices, we realized that making a flange that is highly compliant based on being thin and flexible and that provides a barrier to moisture and heat based on its composition is important to the performance of the bond between the adhesive tape and the flange. Flanges that are relatively thick and stiff result in greater stress concentrations at the interface of the adhesive tape and the flange, resulting in more rapid failure of the bond therebetween. By creating flanges that are relatively thin and flexible, we can better match the bulk modulus of the flange to the adhesive tape, resulting in better stress distribution between the flange and the adhesive tape and improved performance of the bond and allowing better contouring of the flange and the adhesive tape around the nose of a patient as the adhesive tape is applied. This helps to protect noses of patients from irritation by movement of the nasal tube device, and helps to prevent pressure wounds and erosion of nostrils of the patients. By making the flanges from materials that provide a barrier to moisture and heat, we also can protect the adhesive tape from warm, humid breath exhaled by patients, offering further improvements in adhesive performance.

Given the performance of the bond between the adhesive tape and the flange, and the adhesive tape and the skin, we also realized that we would not need to rely on interference pressure, created by the use of round nasal tubes having a circular tubular cross section being forced through a narrow non-round nasal cavity, to promote retention of the nasal tubes. Rather, we could use nasal tubes having an elliptical tubular cross section that is noncircular that better matches the internal anatomy of the nasal passage, resulting in substantially lower contact forces with the nasal passage tissues. Our nasal tube with an elliptical tubular cross section that is noncircular better matches the anatomy of the nasal passages of patients than does a comparably sized nasal tube with a circular cross section. This makes our nasal tube easier to insert into a nasal passage, resulting in less tissue disturbance and less interference with nasal tissue, which decreases the risk of injury and bleeding associated with insertion and advancement of the nasal tube through the nasal passage.

The process of placing and securing our nasal tube devices is also quicker and easier than for the nasal tube device of U.S. Pat. No. 10,426,941 because there is no flexible line requiring an invasive placement procedure, and removal of our nasal tube devices from patients does not requires a tool, but rather can be done by hand.

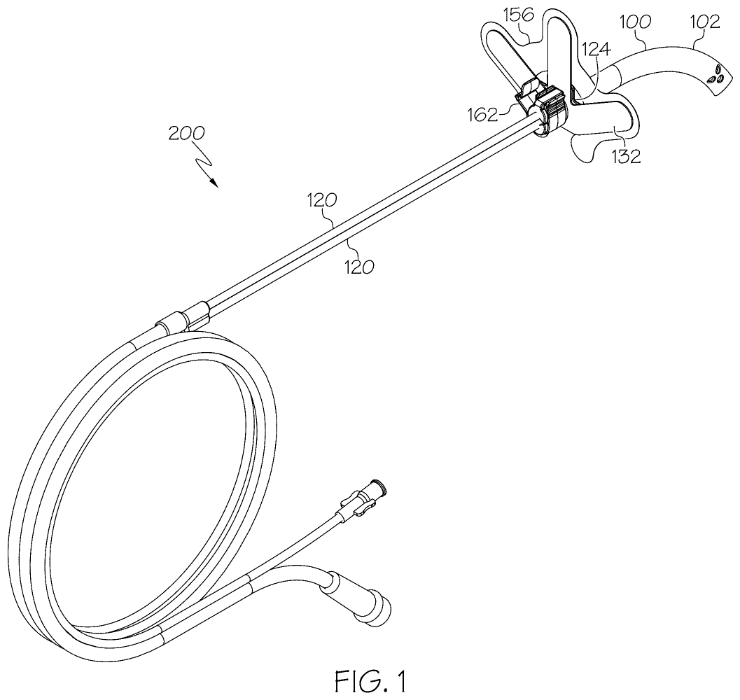

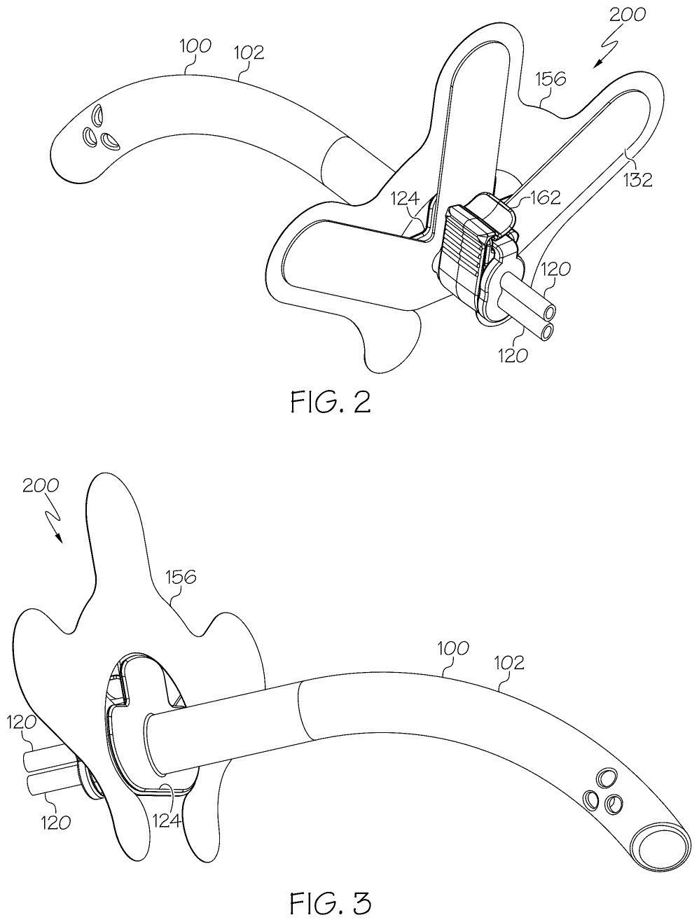

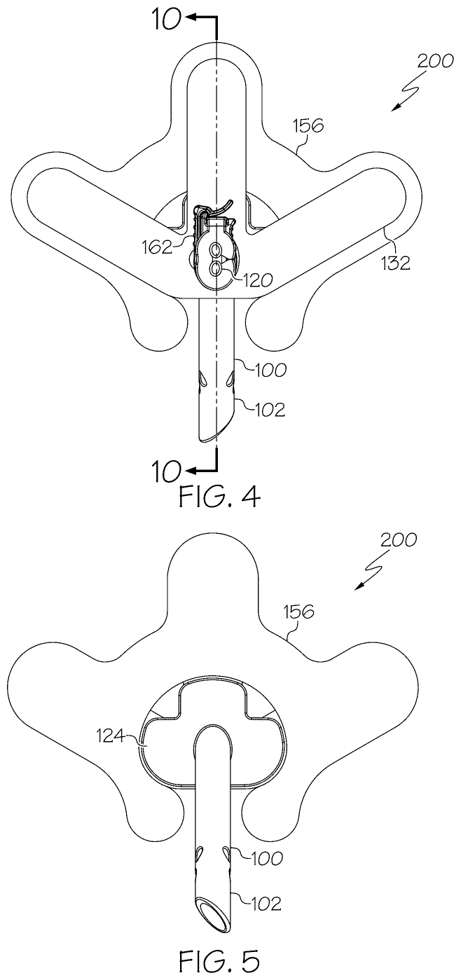

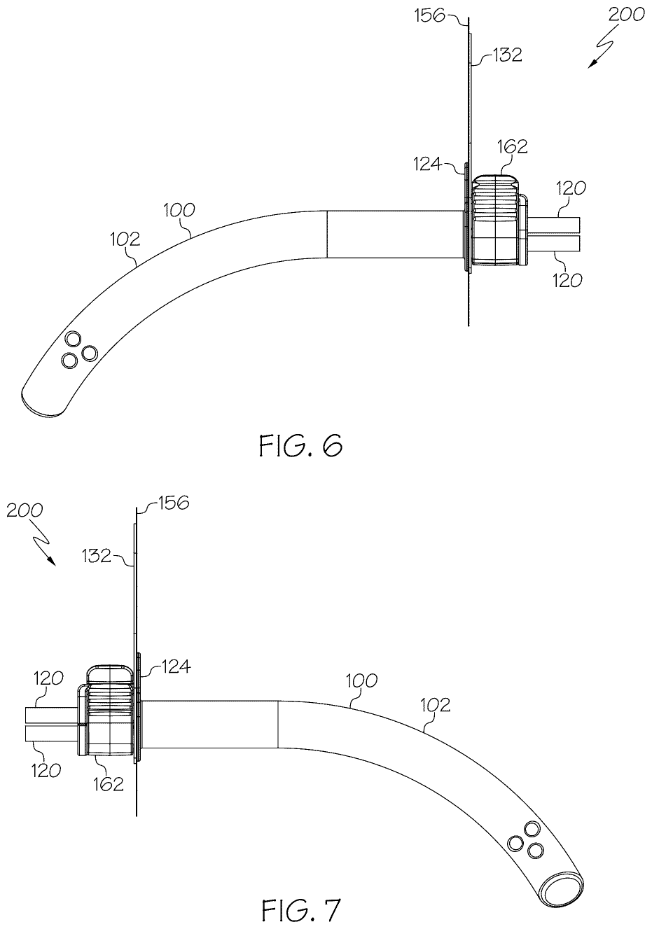

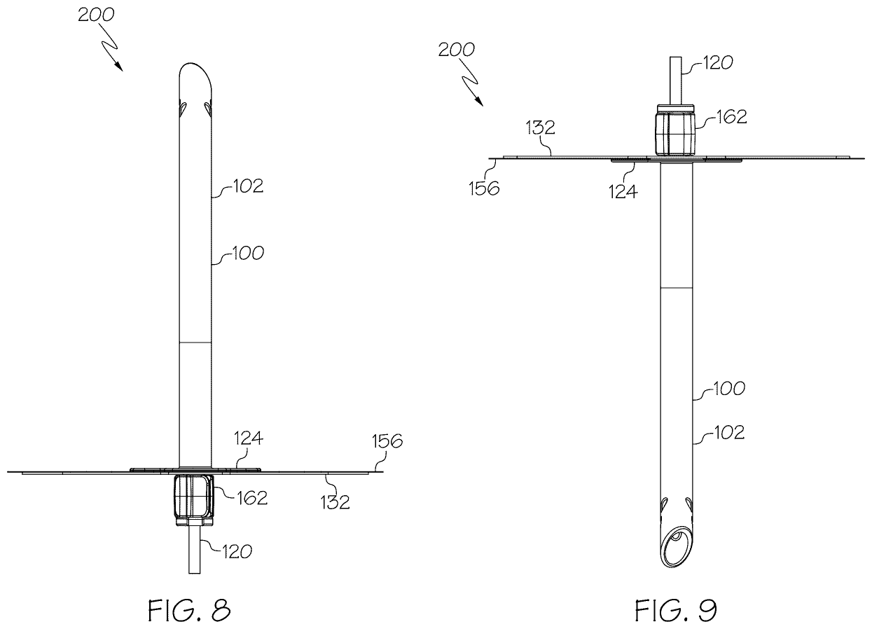

Considering our nasal tube device comprising (a) a nasal tube, (b) a flange, (c) an adhesive tape, and (d) a clip, as described above, in more detail, FIGS. 1-10 show an embodiment of our nasal tube device 100 comprising (a) a nasal tube 102, (b) a flange 124, (c) an adhesive tape 132, and (d) a clip 162 that can be used for securing a nasal tube 102 and one or more other tubes 120 to a nose of a patient, as part of a system 200 comprising the nasal tube device 100 and the one or more other tubes 120.

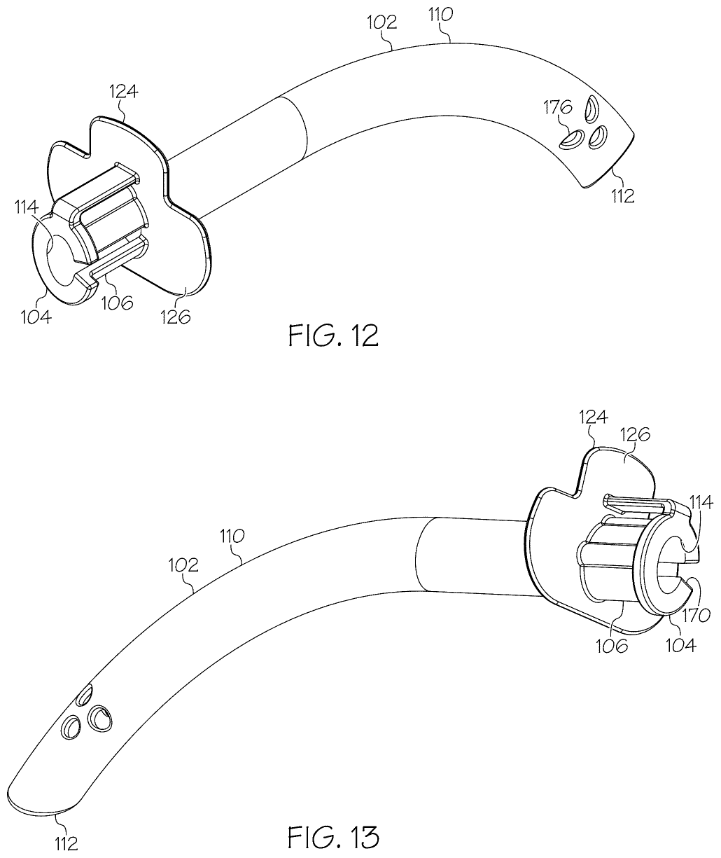

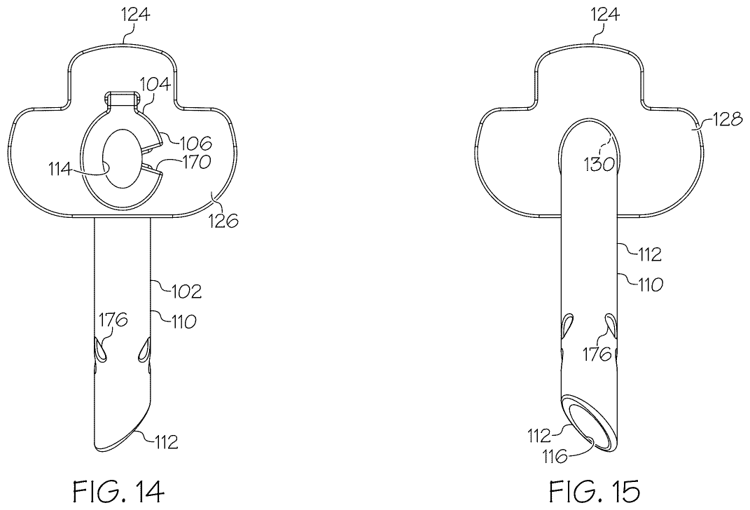

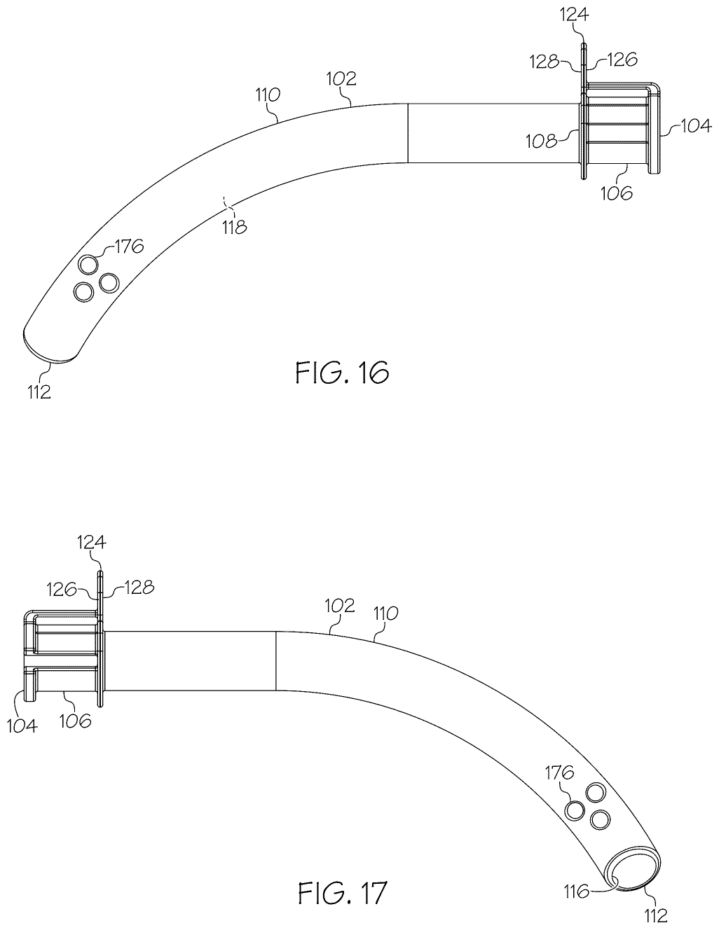

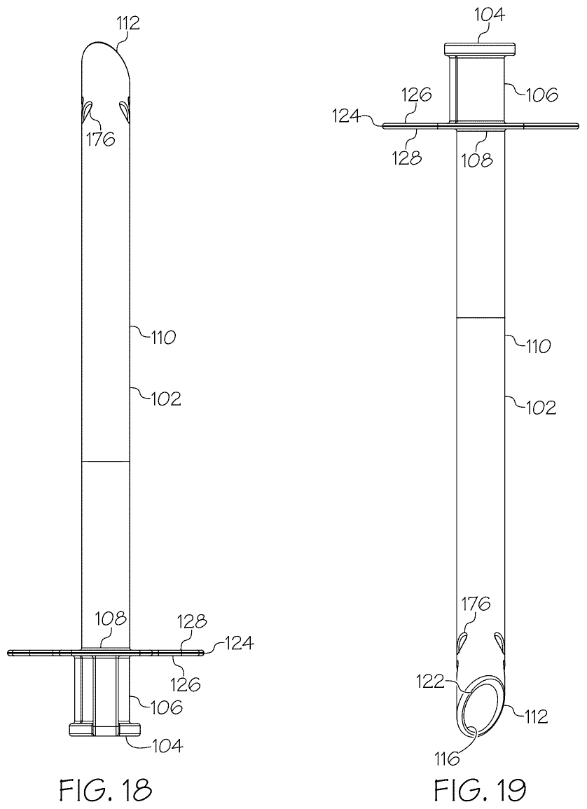

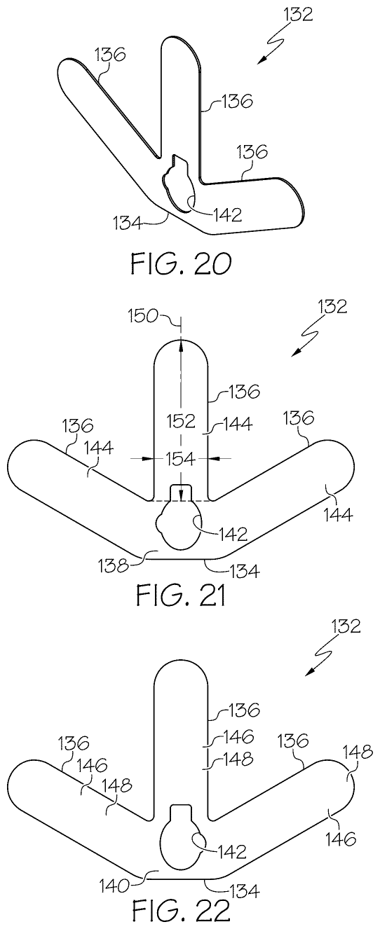

As shown in FIG. 11, with reference to FIGS. 12-19, the nasal tube device 100 comprises a nasal tube 102.

The nasal tube 102 can be any kind of suitable nasal tube. The nasal tube 102 can be, for example, a nasopharyngeal airway, which can be used, among other things for critical airway management of a patient. The nasal tube 102 also can be, for example, a nasotracheal tube, which can be used, among other things, to provide a secure airway for patients who require mechanical ventilation. The nasal tube 102 also can be, for example, a nasogastric tube, which can be used, among other things, to deliver liquids, food, and medications to the stomach of a patient or to remove substances from the stomach of the patient. The nasal tube 102 also can be, for example, a nasojejunal tube, which can be used, among other things, to deliver nutrients to the small intestine of a patient.

Accordingly, in some embodiments the nasal tube 102 comprises a nasopharyngeal airway, a nasogastric tube, or a nasojejunal tube.

As shown in FIGS. 12-19, the nasal tube 102 comprises, sequentially, a proximal end 104, a proximal-clip portion 106, a proximal-flange portion 108, a distal portion 110, and a distal end 112.

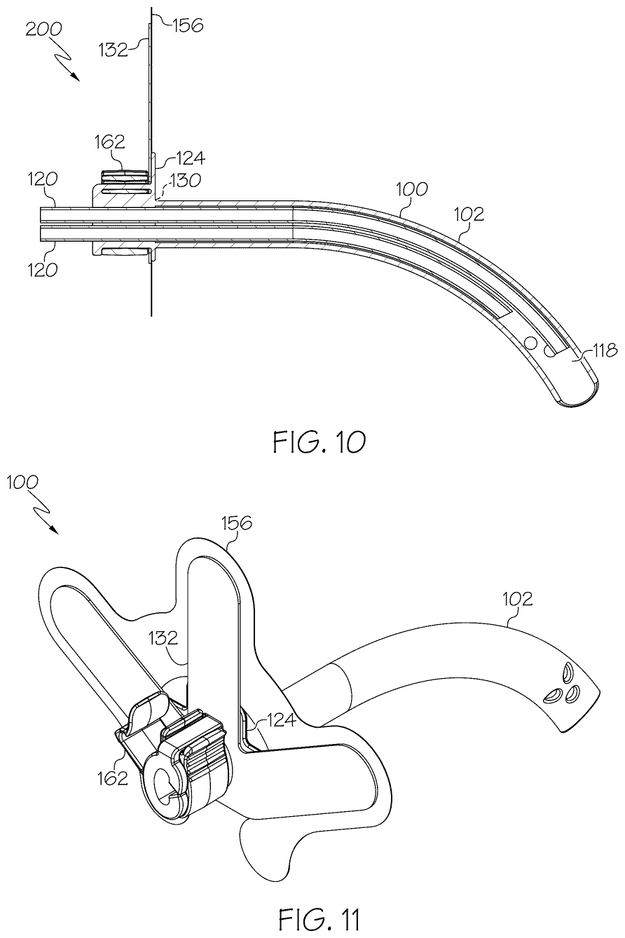

As shown in FIGS. 12-19, with reference to FIG. 10, the proximal end 104 of the nasal tube 102 has a proximal opening 114, the distal end 112 of the nasal tube 102 has a distal opening 116, and the nasal tube 102 has a nasal tube lumen 118 extending from the proximal opening 114 of the nasal tube 102 to the distal opening 116 of the nasal tube 102. The nasal tube lumen 118 can have an inner diameter sufficiently large to fit one or more other tubes 120, and can used for placement of the one or more other tubes 120. For example, for a nasal tube 102 that comprises a nasopharyngeal airway for providing airway patency, the nasal tube lumen 118 can have an inner diameter sufficiently large to fit a tube for oxygen delivery, such as an oxygen delivery line, to provide retropalatal delivery of oxygen, and/or a tube for carbon dioxide monitoring, such as a capnography sampling line, for retropalatal sampling of exhaled breath for capnography monitoring, among other tubes. Also for example, the nasal tube lumen 118 of the nasopharyngeal airway can be used for placement of the tube for oxygen delivery and/or the tube for carbon dioxide monitoring.

As shown in FIG. 19, the nasal tube 102 has an elliptical tubular cross section 122 that is noncircular. Accordingly, the nasal tube 102 has a tubular cross section that has a generally ovoid shape rather than, for example, a generally circular shape. The elliptical tubular cross section 122 of the nasal tube 102 can be elliptical and noncircular, for example, based on the cross section having two foci, and the sum of the distances from each point on the cross section to the two foci being identical, without the cross section forming a circle. The elliptical tubular cross section 122 of the nasal tube 102 also can be elliptical and noncircular, for example, based on the tubular cross section of the nasal tube having two axes of symmetry, a longer major axis and a shorter minor axis, with the major and minor axes intersecting at the center of the cross section, the major axis connecting the two vertices of the cross section, and the minor axis connecting the two co-vertices of the cross section. As discussed above, a nasal tube 102 having an elliptical tubular cross section 122 that is noncircular is advantageous based on better matching the anatomy of the nasal passages of patients in comparison to a comparably sized nasal tube with a circular tubular cross section, making the nasal tube 102 easier to insert into a nasal passage and decreasing the risk of epistaxis and trauma to the tissues of the nasal cavity. To match the elliptical tubular cross-section 122 to the anatomy of the nasal passages of patients, the distance between foci on the major axis, known as the interfocal length, should be between 0.50 to 0.99 times the length of the major axis. To accommodate positioning of the one or more other tubes 120 within the nasal tube lumen 118 of the nasal tube 102, an interfocal length between 0.50 to 0.90 times the length of the major axis is preferable, with an interfocal length of 0.70-0.75 times the length of the major axis being most preferred.

Accordingly, in some embodiments the nasal tube 102 has an elliptical tubular cross section 122 that is noncircular based on the elliptical tubular cross section 122 having a major axis length and an interfocal length, and the interfocal length being 0.50 to 0.99 times the major axis length, 0.50 to 0.90 times the major axis length, or 0.70 to 0.75 times the major axis length.

As shown in FIG. 12 and FIGS. 14-19, the nasal tube 102 can be provided with one or more ventilation holes 176.

The nasal tube 102 should be made from a material that provides for the nasal tube 102 to be sufficiently flexible to conform to the anatomy of a patient, but not so soft that the nasal tube 102 cannot maintain patency during placement and normal ambulation. Suitable properties for materials for making the nasal tube 102 include, for example, (i) a tensile strength of 2.0 to 20 MPa, a tensile modulus of 0.5 to 10 MPa, and a hardness of 50 Shore-00 to 90 Shore A, (ii) a tensile strength of 5 to 15 MPa, a tensile modulus of 1 to 5 MPa, and a hardness of 30 to 70 Shore A, or (iii) a tensile strength of 8 to 10 MPa, a tensile modulus of 2 to 3 MPa, and a hardness of 40 to 60 Shore A. The nasal tube 102 may be made from, for example, one or more of silicone rubber elastomer, natural latex rubber, thermoplastic elastomer, styrene block copolymer, thermoplastic polyolefin, thermoplastic polyurethane, thermoplastic vulcanizate, ethylene-propylene-diene-monomer, copolymer derivatives of ethylene-propylene-diene-monomer, styrene-butadiene-rubber, copolymer derivatives of styrene-butadiene-rubber, flexible non-elastomeric polymer, or plasticized polyvinyl chloride. These materials provide for the nasal tube 102 to be sufficiently flexible to conform to the anatomy of a patient, but not so soft that the nasal tube 102 cannot maintain patency during placement and normal ambulation. Silicone rubber elastomer is particularly preferred because in addition to providing sufficient flexibility without being too soft, silicone rubber elastomer has good biocompatibility. Moreover, for a nasal tube 102 corresponding to a nasopharyngeal airway and the use of one or more other tubes 120 including a tube for oxygen delivery, where combustion is a potential concern, silicone rubber elastomers are desirable because they generally exhibit high ignition temperatures, low flame spread, and good resistance to oxidation

A lubricant may be applied to one or more portions of surfaces of the nasal tube 102 to reduce friction during placement.

As shown in FIG. 11, with reference to FIGS. 12-19, the nasal tube device 100 also comprises a flange 124. As shown in FIGS. 12-19, with reference to FIG. 10, the flange 124 has a proximal face 126 and a distal face 128. The flange 124 extends radially outwardly from the proximal-flange portion 108 of the nasal tube 102. The nasal tube 102 extends through the flange 124.

The flange 124 can be formed integrally to the nasal tube 102 at the proximal-flange portion 108 of the nasal tube 102, for example based on the flange 124 being made from the same material as the nasal tube 102 and being molded as a part of the nasal tube 102 at the proximal-flange portion 108 of the nasal tube 102. Alternatively, the flange 124 can be formed separately from the nasal tube 102, and attached to the nasal tube 102 after the nasal tube 102 and the flange 124 have been formed. In this case, the flange 124 can made having a hole 130 therethrough, and the flange 124 can be attached to the nasal tube 102 by placing the nasal tube 102 through the hole 130 of the flange 124 at the distal end 112 of the nasal tube 102 or the proximal end 104 of the nasal tube 102, such that the nasal tube 102 extends through the hole 130 of the flange 124, and then sliding the flange 124 to the proximal-flange portion 108 of the nasal tube 102. In this case the flange 124 also optionally can be fixed to the nasal tube 102 at the proximal-flange portion 108 of the nasal tube 102, for example by use of an adhesive.

Accordingly, in some embodiments the flange 124 is formed integrally to the nasal tube 102 at the proximal-flange portion 108 of the nasal tube 102. Also in some embodiments the flange 124 is formed separately from the nasal tube 102, the flange 124 has a hole 130 therethrough, and the nasal tube 102 extends through the hole 130 of the flange 124.

The flange 124 is highly compliant based on being thin and flexible and provides a barrier to moisture and heat based on its composition. Like the nasal tube 102, the flange 124 should be made from a material that provides for the flange 124 to be sufficiently flexible to conform to the anatomy of a patient. Suitable thicknesses for the flange 124 include, for example, 0.50 to 5.00 mm, 0.60 to 2.50 mm, or 0.75 to 1.00 mm. Suitable properties for materials for making the flange 124 include, for example, (i) a tensile strength of 2.0 to 20 MPa, a tensile modulus of 0.5 to 10 MPa, and a hardness of 50 Shore-00 to 90 Shore A, (ii) a tensile strength of 5 to 15 MPa, a tensile modulus of 1 to 5 MPa, and a hardness of 30 to 70 Shore A, or (iii) a tensile strength of 8 to 10 MPa, a tensile modulus of 2 to 3 MPa, and a hardness of 40 to 60 Shore A. Suitable materials for making the flange 124, include, for example, silicone rubber elastomer, natural latex rubber, thermoplastic elastomer, styrene block copolymer, thermoplastic polyolefin, thermoplastic polyurethane, thermoplastic vulcanizate, ethylene-propylene-diene-monomer, copolymer derivatives of ethylene-propylene-diene-monomer, styrene-butadiene-rubber, copolymer derivatives of styrene-butadiene-rubber, flexible non-elastomeric polymer, or plasticized polyvinyl chloride. As discussed above, making a flange 124 that is highly compliant based on being thin and flexible and that provides a barrier to moisture and heat based on its composition is important to the performance of the bond between the adhesive tape 132 and the flange 124, because creating flanges 124 that are relatively thin and flexible allows better matching the bulk modulus of the flange 124 to the adhesive tape 132, resulting in better stress distribution between the flange 124 and the adhesive tape 132 and improved performance of the bond and allowing better contouring of the flange 124 and the adhesive tape 132 around the nose of a patient as the adhesive tape 132 is applied, and because making the flanges 124 from materials that provide a barrier to moisture and heat allows protection of the adhesive tape 132 from warm, humid breath exhaled by patients, offering further improvements in adhesive performance.

Accordingly, in some embodiments the flange 124 has a thickness of 0.50 to 5.00 mm, 0.60 to 2.50 mm, or 0.75 to 1.00 mm.

Also in some embodiments the flange 124 is made from a material having (i) a tensile strength of 2.0 to 20 MPa, a tensile modulus of 0.5 to 10 MPa, and a hardness of 50 Shore-00 to 90 Shore A, (ii) a tensile strength of 5 to 15 MPa, a tensile modulus of 1 to 5 MPa, and a hardness of 30 to 70 Shore A, or (iii) a tensile strength of 8 to 10 MPa, a tensile modulus of 2 to 3 MPa, and a hardness of 40 to 60 Shore A.

Also in some embodiments the flange 124 is made from one or more of silicone rubber elastomer, natural latex rubber, thermoplastic elastomer, styrene block copolymer, thermoplastic polyolefin, thermoplastic polyurethane, thermoplastic vulcanizate, ethylene-propylene-diene-monomer, copolymer derivatives of ethylene-propylene-diene-monomer, styrene-butadiene-rubber, copolymer derivatives of styrene-butadiene-rubber, flexible non-elastomeric polymer, or plasticized polyvinyl chloride.

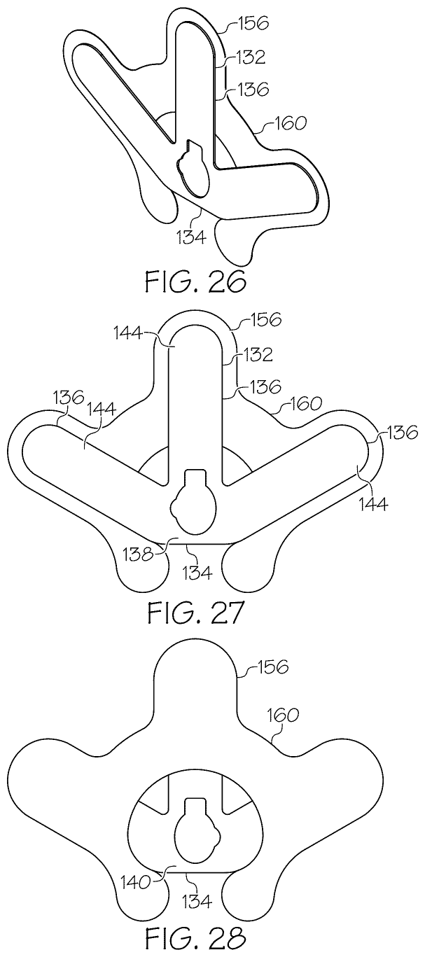

As shown in FIG. 11, with reference to FIGS. 22-28, the nasal tube device 100 also comprises an adhesive tape 132. As shown in FIGS. 22-28, the adhesive tape 132 comprises a base portion 134 and one or more strip portions 136 extending from the base portion 134. The base portion 134 of the adhesive tape 132 has a proximal side 138, a distal side 140, and a hole 142 therethrough. Each of the one or more strip portions 136 have a proximal side 144 and a distal side 146. The distal side 146 of each of the one or more strip portions 136 each have an adhesive surface 148.

The nasal tube 102 extends through the hole 142 of the base portion 134 of the adhesive tape 132. Similarly as for a flange 124 formed separately from the nasal tube 102, this can be accomplished for example by placing the nasal tube 102 through the hole 142 of the base portion 134 of the adhesive tape 132 at the distal end 112 of the nasal tube 102 or the proximal end 104 of the nasal tube 102, such that the nasal tube 102 extends through the hole 142 of the base portion 134 of the adhesive tape 132, and then sliding the adhesive tape 132 to the proximal-flange portion 108 of the nasal tube 102.

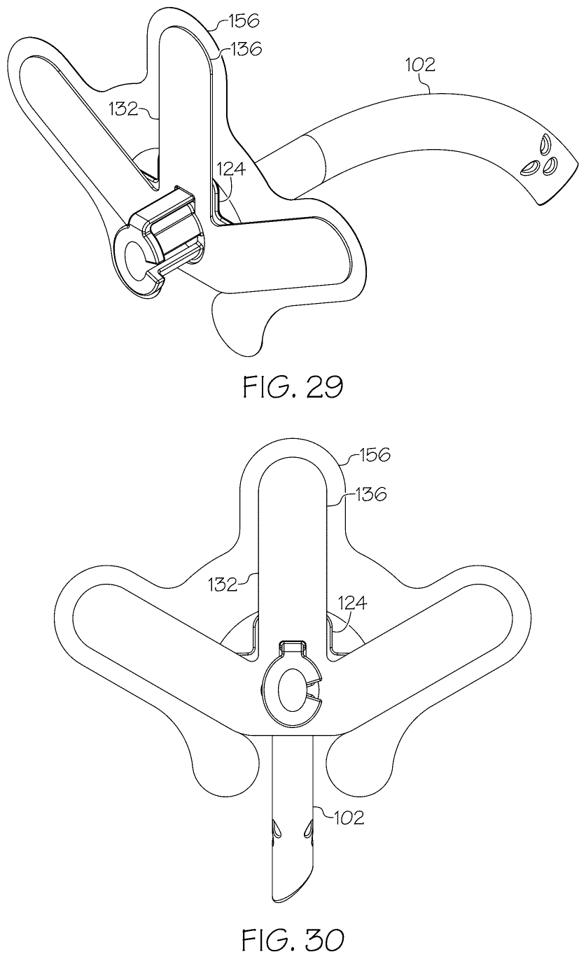

The adhesive tape 132 is attached to the flange 124 at the base portion 134 of the adhesive tape 132. For example, as shown in FIGS. 29-30, the adhesive tape 132 can be attached to the flange 124 based on the distal side 146 of the base portion 134 of the adhesive tape 132 being adhered to the proximal face 126 of the flange 124. In these examples, the attachment of the adhesive tape 132 to the flange 124 can be accomplished, for example, by use of a same adhesive that is present at the adhesive surfaces 148 of the distal side 146 of each of the one or more strip portions 136 that is also present on the distal side 140 of the base portion 134 of the adhesive tape 132 and/or based on a secondary adhesive present on the distal side 140 of the base portion 134 of the adhesive tape 132.

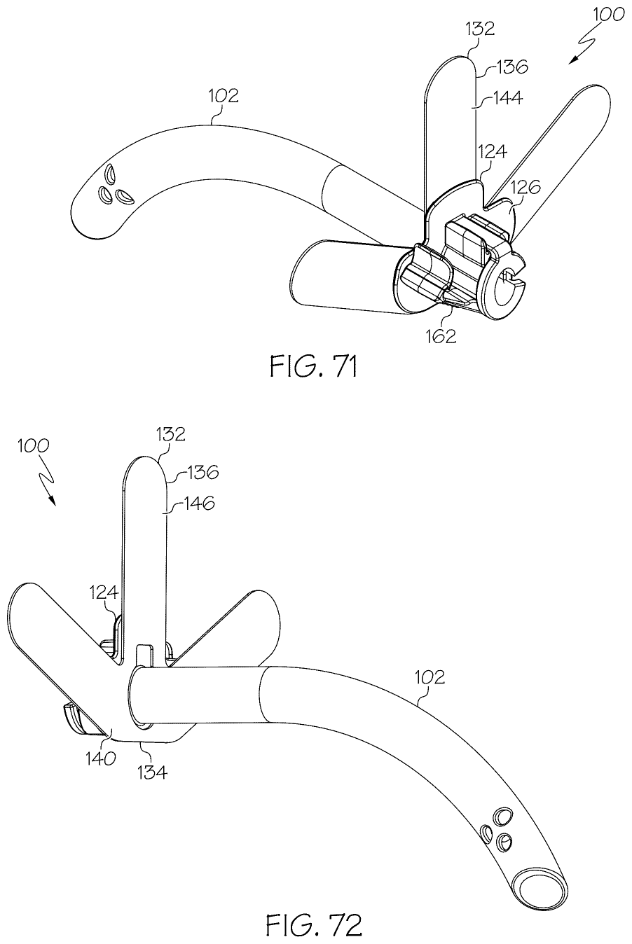

Also for example, as shown in FIGS. 71-72, the adhesive tape 132 can be attached to the flange 102 based on the proximal side 138 of base portion 134 of the adhesive tape 132 being adhered to the distal face 128 of the flange 124. In these examples, the attachment of the adhesive tape 132 to the flange 124 can be accomplished, for example, by use of a secondary adhesive present on the proximal side 138 of the base portion 134 of the adhesive tape 132.

For the nasal tube device 100 comprising a flange 124 that is formed integrally to the nasal tube 102 at the proximal-flange portion 108 of the nasal tube 102, the adhesion of the adhesive tape 132 to the flange 124 can be accomplished after the nasal tube 102 has been placed through the hole 142 of the base portion 134 of the adhesive tape 132 and the adhesive tape 132 has been slid to the proximal-flange portion 108 of the nasal tube 102. For the nasal tube device 100 comprising a flange 124 that is formed separately from the nasal tube 102, the adhesion of the adhesive tape 132 to the flange 124 can be accomplished either before or after the nasal tube 102 has been placed through the hole 142 of the base portion 134 of the adhesive tape 132 and the hole 142 of the flange 124 and the adhesive tape 132 and the flange 124 have been slid to the proximal-flange portion 108 of the nasal tube 124.

In either of these cases and in other cases, the attachment can be accomplished by use of one or more adhesives suitable for adhering the adhesive tape 132 to the flange 124, for example based on the adhesive being pre-selected for chemical compatibility with the material from which the flange 124 is made. For simplicity of manufacturing the adhesive tape 132, an adhesive pre-selected for safe use on faces of patients may be used for adhering the adhesive tape 132 to the flange 124 as well as securing the adhesive tape 132 to the face of a patient.

Adhesives suitable for adhering the adhesive tape 132 to the flange 124 include, for example, silicone adhesives, acrylate adhesives, and synthetic rubber adhesives. Adhesive selection must consider various aspects such as (i) potential biological response, including irritation/sensitization, (ii) tack, including the ability to rapidly form a bond with the substrate and to resist debonding, and (iii) bond strength, including the ability to resist tensile, sheer, and peel forces once bonded to the substrate. The strength of the adhesive should be appropriately matched to the securement needs and duration of expected use. Silicone adhesives are generally lower strength than acrylic adhesives or synthetic rubber adhesives. Silicone adhesives remove cleanly and without causing injury to the underlying skin. Acrylic adhesives generally offer higher tack and adhesion strength, but are generally more irritating to the skin and may result in minor trauma during removal. Synthetic-rubber adhesives offer the most aggressive tack and highest bond strengths, but this also increases the likelihood of injury with removal. Another concern for an adhesive used to secure a nasal tube is how the adhesive performs in the presence of moisture and heat. The warm, moist air being exhaled through the nostrils of a patient creates a warm and humid environment that can lead to premature failure of some adhesives. Silicone adhesives are inherently more resistant to heat and moisture than synthetic rubber or acrylic adhesives, making silicone adhesives particularly suitable for applications around the nostrils.

Also in either of these cases and in other cases, the attachment can be accomplished during manufacture of the nasal tube device 100 or by an end user prior to use.

Accordingly, in some embodiments the adhesive tape 132 is attached to the flange 124 based on the distal side 140 of base portion 134 of the adhesive tape 132 being adhered to the proximal face 126 of the flange 124. Also in some embodiments the adhesive tape 132 is attached to the flange 124 based on the proximal side 138 of base portion 134 of the adhesive tape 132 being adhered to the distal face 128 of the flange 124.

Alternatively, the adhesive tape 132 can be attached to the proximal face 126 of the flange 124 or the distal face 128 of the flange 124 by mechanical means, such as having one or more raised bosses or grooves on the flange 124 that interface with holes, slots, or tabs on the adhesive tape 132 to mechanically couple the adhesive tape 132 to the flange 124. Further alternatively, the adhesive tape 132 can be attached to the proximal face 126 of the flange 124 or the distal face 128 of the flange 124 by other means, such as overmolding, which involves placing the adhesive tape 132 in a part mold for the flange 124 prior to introducing the material from which the flange 124 will be made, such as a liquid silicone rubber. The liquid silicone rubber can then be pressed into fabric of the adhesive tape 132 during an injection molding process, chemically and mechanically bonding the adhesive tape 132 and the silicone rubber of the flange together when the silicone rubber is cured. Combinations of one or more of these and/or other approaches also can be used.

As discussed above, incorporating an adhesive tape 132 that is pre-attached to the flange 124 and that is pre-selected for material compatibility and skin safety is advantageous because the flange 124 provides a suitable surface space for attachment of the adhesive tape 132, ad-hoc tape selection and poor adhesive bonding between the adhesive tape 132 and the nasal tube 102 are avoided, the adhesive tape 132 can be pre-selected for safe use on the face, avoiding serious skin injuries, and the flange 124 can be provided with sufficient dedicated attachment points for the adhesive tape 132 to decrease the risk of creating pressure points.

As noted above, the distal side 146 of each of the one or more strip portions 136 each have an adhesive surface 148. This can be based, for example, on the presence of one or more suitable adhesives pre-selected for safe use on the faces of patients at each of the adhesive surfaces 148. Examples of suitable adhesives pre-selected for safe use on faces of patients include, for example, silicone adhesives, acrylate adhesives, and synthetic rubber adhesives, as discussed above.

Also as noted above, the adhesive tape 132 comprises one or more strip portions 136 extending from the base portion 134. The one or more strip portions 136 should be sufficiently long and narrow to allow contouring of the flange 124 and the adhesive tape 132 around the nose of a patient as the adhesive tape 132 is applied. Thus, for example, as shown in FIG. 21, the one or more strip portions 136 can each have a major axis 150 having a major axis length 152 and a major axis width 154, with the major axis length 152 being substantially greater than the major axis width 154, for example about two to five times greater, three to four times greater, or about three and one-half times greater.

The use of more than one strip portions 136 can be useful for keeping the nasal tube device 100 secured to the face of the patient during use. Thus, for example, the one or more strip portions 136 of the adhesive tape 132 can comprise two or more strip portions 136 extending from the base portion 134 of the adhesive tape 132, for example, two to four strip portions 136, or three strip portions 136, extending from the base portion 134 of the adhesive tape 132.

Accordingly, in some embodiments the one or more strip portions 136 of the adhesive tape 132 comprise two to four strip portions 136, or three strip portions 136, extending from the base portion 134 of the adhesive tape.

Suitable backing materials for the adhesive tape 132 include, for example, polymeric films made from polyethylene terephthalate, polyethylene, or polyurethane, which are commonly used for adhesive tapes, non-woven polymeric textiles made from polyethylene terephthalate, polyurethane, or cellulose, which also are commonly used, woven textiles made from cotton, which also are commonly used, or foams made from polyethylene/polyvinyl chloride, which also are commonly used.

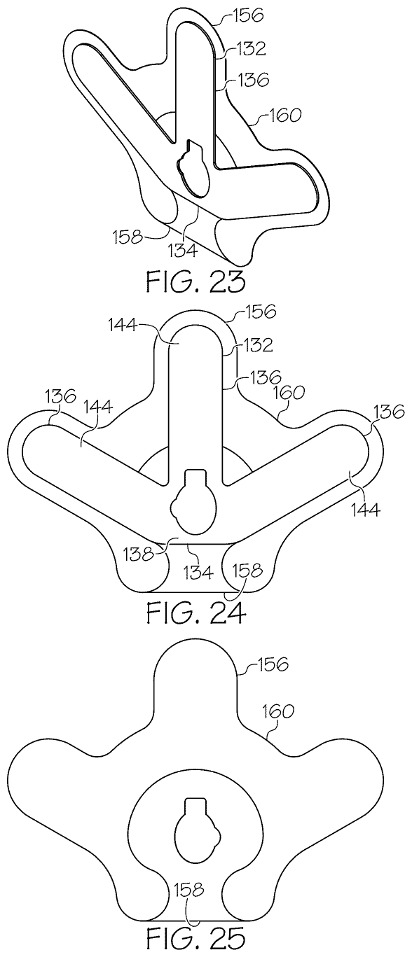

As shown in FIGS. 29-30, with reference to FIGS. 23-28, in some embodiments, the nasal tube device 100 further comprises an adhesive liner 156, the adhesive liner 156 covering the adhesive surfaces 148 of the distal sides 146 of the one or more strip portions 136 of the adhesive tape 132. The adhesive liner 156 can be useful for covering the adhesive for adhering the adhesive tape 132 to the flange 124 prior to attaching the adhesive tape 132 to the flange 124 and/or for covering the adhesive surfaces 148 of the distal side 146 of each of the one or more strip portions 136 prior to applying the adhesive surfaces 148 to the face of a patient. As shown in FIGS. 29-30, the adhesive liner 156 can be provided, for example, in two parts, a first part 158 disposed at the base portion 134 of the adhesive tape 132 that can be removed, for example, just prior to attaching the adhesive tape 132 to the flange 124, and a second part 160 disposed along major portions of the one or more strip portions 136 of the adhesive tape 132 that can be removed, for example, just prior to applying the adhesive surface 148 to the face of the patient. The adhesive liner 156 can be useful for preventing unintended adhesion between the adhesive tape 132 and other surfaces during manufacture and use of the nasal tube device 100.

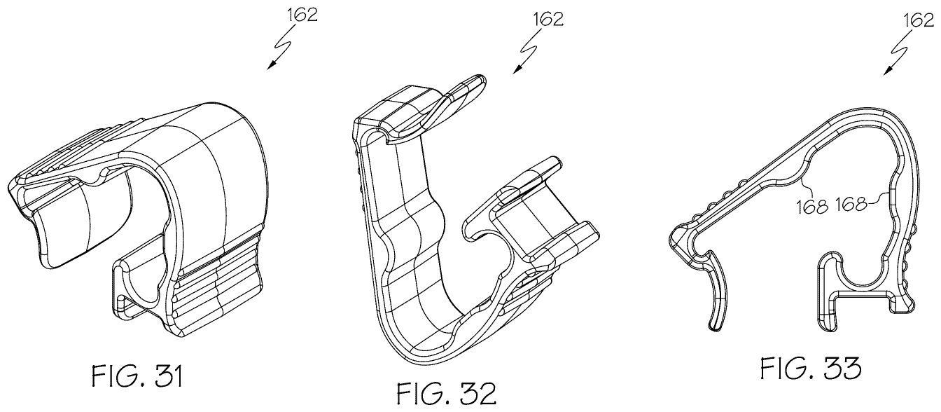

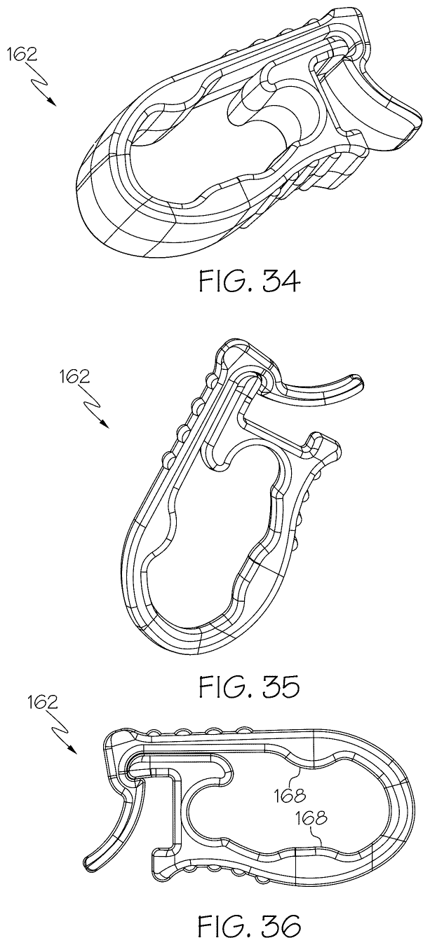

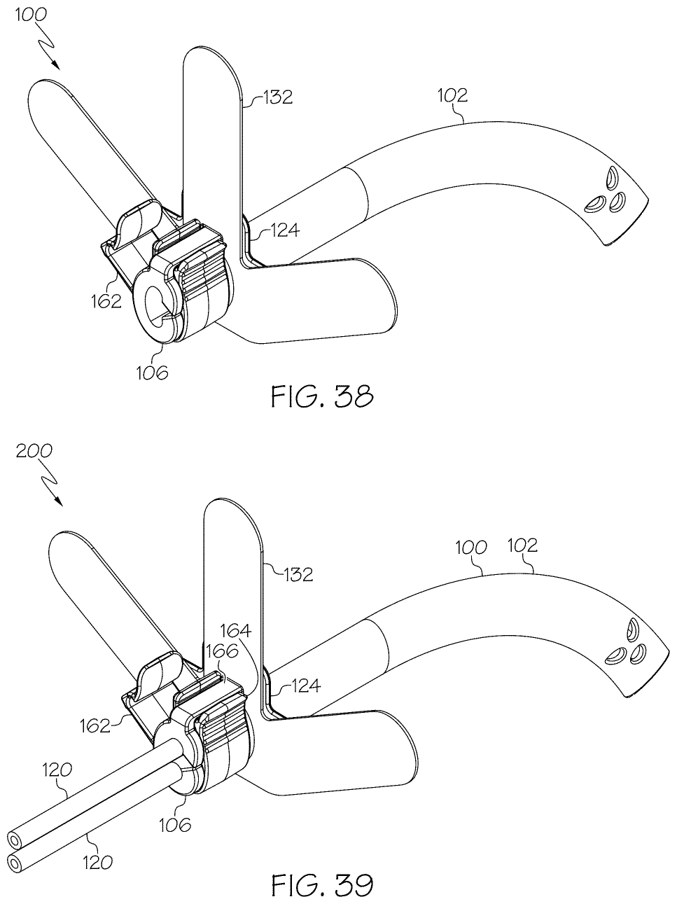

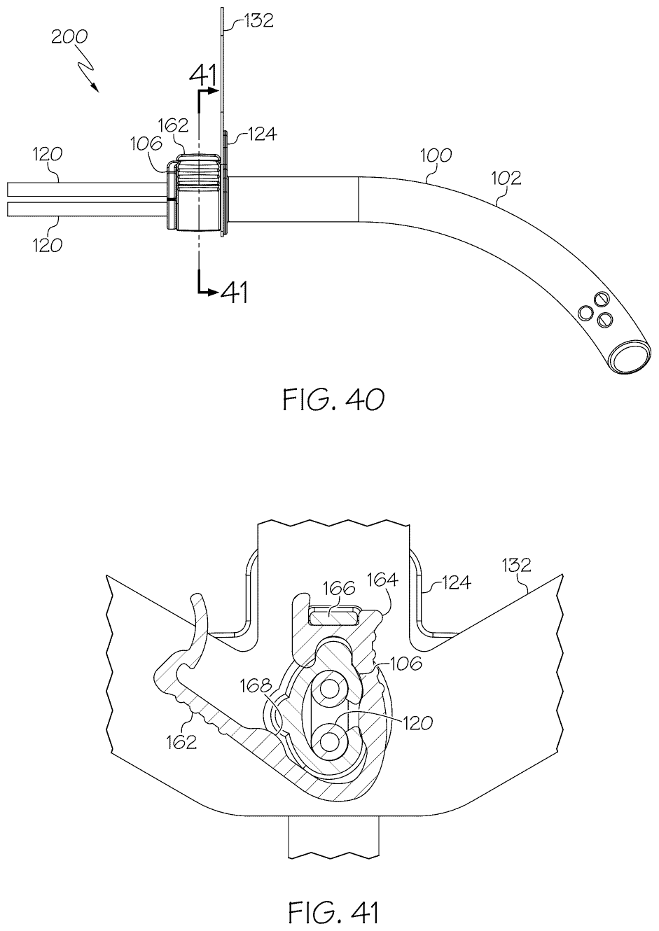

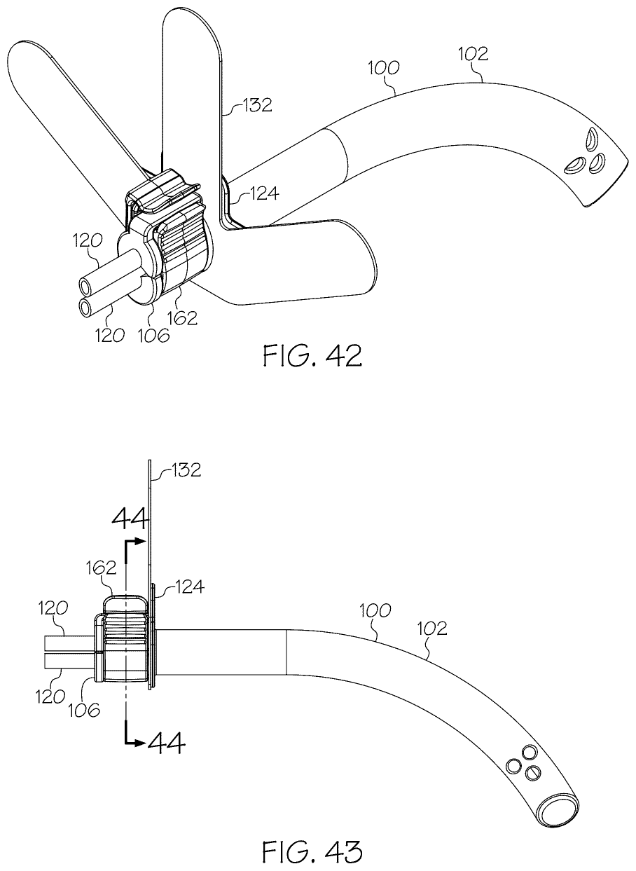

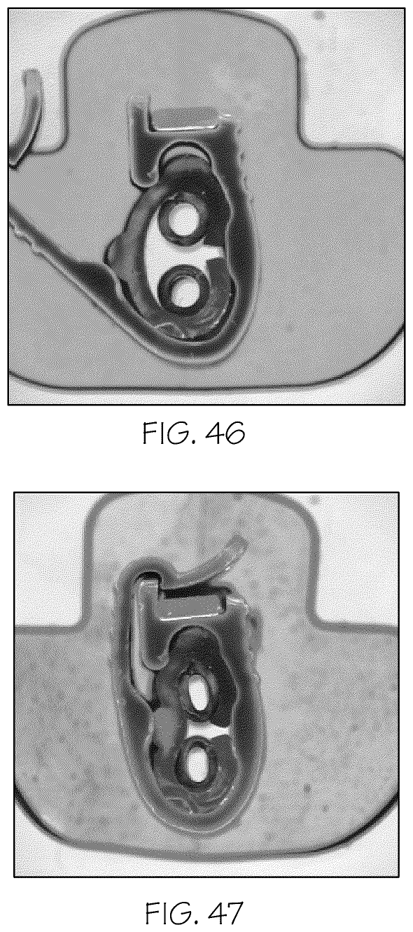

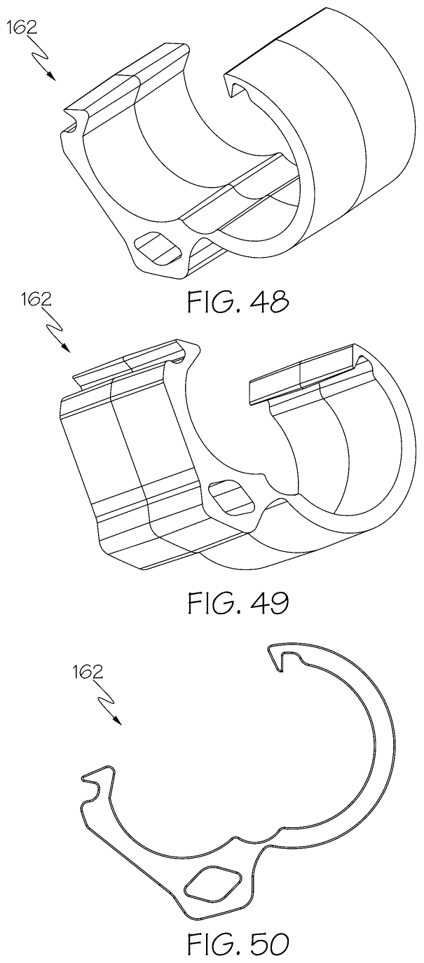

As shown in FIG. 11, with reference to FIGS. 31-36, the nasal tube device 100 also comprises a clip 162 for securing one or more other tubes 120 to the nasal tube 102 within the nasal tube lumen 118 of the nasal tube 102 at the proximal-clip portion 106 of the nasal tube 102. As shown in schematically in FIGS. 38-44 and in micrographs of cross sections of a prototype of the nasal tube device 100 in FIGS. 45-47, the clip 162 is reversibly closeable about the proximal-clip portion 106 of the nasal tube 102 to reversibly compress the proximal-clip portion 106 of the nasal tube 102 about the one or more other tubes 120 when closed. This is useful for holding the one or more other tubes 120 in place within the nasal tube lumen 118 of the nasal tube 102 when the clip 162 is closed, and thus for preventing displacement of the one or more other tubes 120.

As shown in FIG. 39 and FIG. 41, the clip 162 and the proximal-clip portion 106 of the nasal tube 102 can be made with structures 164, 166 that provide a complementary fit between the clip 162 and the proximal-clip portion 106 of the nasal tube 102 when the clip 162 is closed about the proximal-clip portion 106 of the nasal tube 102. This can be useful for preventing the clip 162 from slipping free of proximal-clip portion 106 of the nasal tube 102 when closed.

Accordingly, in some embodiments the clip 162 and the proximal-clip portion 106 of the nasal tube 102 comprise structures 166, 168 that provide a complementary fit between the clip 162 and the proximal-clip portion 106 of the nasal tube 102 when the clip 162 is closed about the proximal-clip portion 106 of the nasal tube 102.

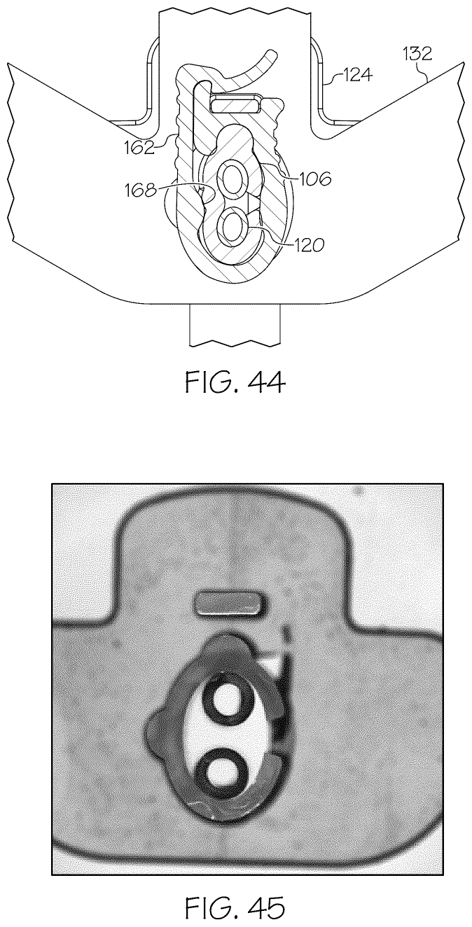

Also, as shown in FIG. 41 and FIG. 44, with reference to FIGS. 33-36, one or more of the clip 162 or the proximal-clip portion 106 of the nasal tube 102 can comprise one or more protrusions 168 to reversibly compress the proximal-clip portion 106 of the nasal tube 102 about the one or more other tubes 120 when the clip 162 is closed about the proximal-clip portion 106 of the nasal tube 102. This can promote holding of the one or more other tubes 120 in place within the nasal tube lumen 118 of the nasal tube 102 when the clip 162 is closed, and thus prevention of displacement of the one or more other tubes 120.

Accordingly, in some embodiments one or more of the clip 162 or the proximal-clip portion 106 of the nasal tube 102 comprise one or more protrusions 168 to reversibly compress the proximal-clip portion 106 of the nasal tube 102 about the one or more other tubes 120 when the clip 162 is closed about the proximal-clip portion 108 of the nasal tube 102.

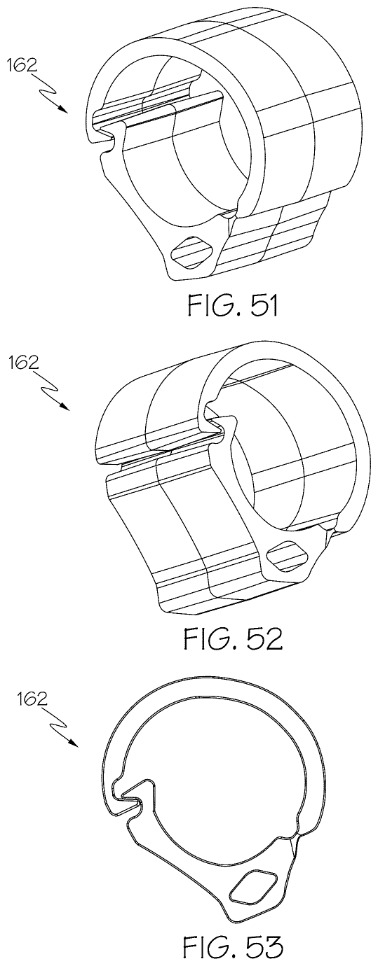

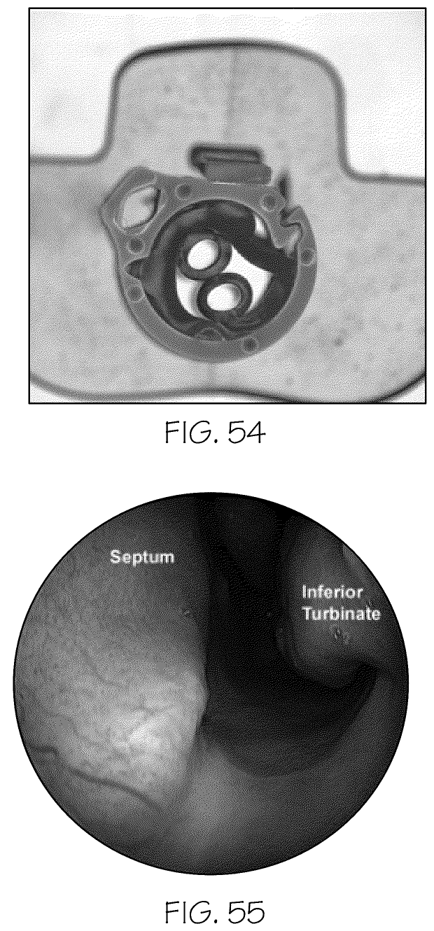

As shown schematically in FIGS. 48-53 and in a micrograph of a cross section of a prototype of the nasal tube device 100 in FIGS. 54, the clip 162 and the proximal-clip portion 106 of the nasal tube 102 also can be provided without any such protrusions and can still hold the one or more other tubes 120 in place within the nasal tube lumen 118 of the nasal tube 102 when the clip 162 is closed.

Optionally, the clip 162 can be attached to the proximal-clip portion 106 of the nasal tube 102, such that the clip 162 remains attached to the nasal tube 102 even when the clip 162 is open. For example, a portion of the clip 162 can be adhered to the proximal-clip portion 106 of the nasal tube 102. This can be useful for preventing an end user from inadvertently dropping the clip 162 during handling and for preventing a patient from inadvertently swallowing the clip 162 if dropped.

Accordingly, in some embodiments, the clip 162 is attached to the proximal-clip portion 106 of the nasal tube 102.

Also optionally, as shown in FIGS. 12-14, the proximal-clip portion 106 of the nasal tube 102 can include a slit 170 along the proximal-clip portion 106 so that the proximal-clip portion 106 is reversibly closeable about the one or more other tubes 120 when the clip 162 is closed about the proximal-clip portion 106.

The clip 162 should be made from a material that provides a blend of strength, stiffness, reasonable cost, availability, and case of manufacturing. Suitable materials for making the clip 162 include, for example, polypropylene, polyethylene, polystyrene, acrylonitrile-styrene, cyclic-olefin-copolymer, polyester-terephthalate, polycyclohexylenedimethylene-terepthalate, copolyester, acrylonitrile-butadiene-styrene, polyacetal, polycarbonate, polyvinylchloride, polylactic-acid, polyimide, polyamide, polyetherimide, polysulfone, polyethersulfone, liquid crystal polymer, polymethyl-methacrylate, polyphthalamide, polyphenylene-oxide, polyphenylene-sulfide, polyphenylene sulfone, syndiotactic-polystyrene, polyaryletherketone, and polyetheretherketone. Of these, materials such as polypropylene, acrylonitrile-styrene, acrylonitrile-butadiene-styrene, and polyamide are preferred for making the clip 162 for their blend of strength, stiffness, cost, availability, and ease of processing. Thermoplastics are generally preferable over thermosets for ease of manufacturing, but thermoset materials offering similar mechanical properties, such as unsaturated polyester, epoxy, or phenolic resins also can be equally suitable for making the clip 162.

Considering the nasal tube 102 in more detail, in addition to the nasal tube 102 having an elliptical tubular cross section 122 that is noncircular, the shape of the distal end 112 of the nasal tube 102 may affect the difficulty or case of passage through the nasal cavity of a patient. Nearly all currently existing nasopharyngeal airways have a beveled tip. Some have the bevel oriented to one side, while others have the bevel oriented such that it faces the floor of the nasal cavity during insertion. Regardless of bevel orientation, a result of the single bevel design is a relatively sharp point at the distal end of a nasal tube. This pointed end can snag on protruding nasal tissue, such as the inferior turbinate, and may become folded back during insertion, increasing resistance and leading to a higher likelihood of trauma and/or bleeding.

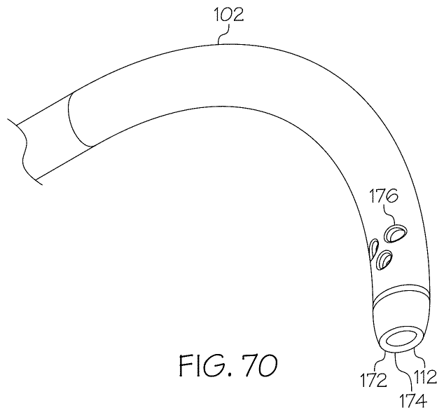

To address these issues associated with beveled tips, as shown in FIG. 70, our nasal tube device 100 can be provided including an axisymmetric tapered tip 172 at the distal end 112 of the nasal tube 102. Unlike a beveled tip that is cut on a bias and terminates in a point that can snag tissues during insertion, the axisymmetric tapered tip 172 offers less potential for snagging tissue due to being symmetrically tapered. As shown in FIG. 70, the axisymmetric tapered tip 172 may be produced, for example, having a perforate distal terminus 174 and one or more ventilation holes 176. Alternatively, the axisymmetric tapered tip 172 may be produced having a perforate distal terminus 174 and without ventilation holes. Also alternatively, the axisymmetric tapered tip 172 may be produced having an imperforate distal terminus 178 and one or more ventilation holes 176. A nasal tube 102 having an axisymmetric tapered tip 172 at the distal end 112 of the nasal tube 102 advantageously can be resistant to impaction and snagging on anatomical perturbances during insertion and advancement through the nasal cavity.

When applied in our nasal tube device 100 comprising a nasal tube 102 having an elliptical tubular cross section 122 that is noncircular, the axisymmetric tapered tip 172 can make our nasal tube 102 even easier to insert into a nasal passage, resulting in even less tissue disturbance and less interference with nasal tissue, and can further decreases the risk of epistaxis and trauma to the tissues of the nasal cavity.

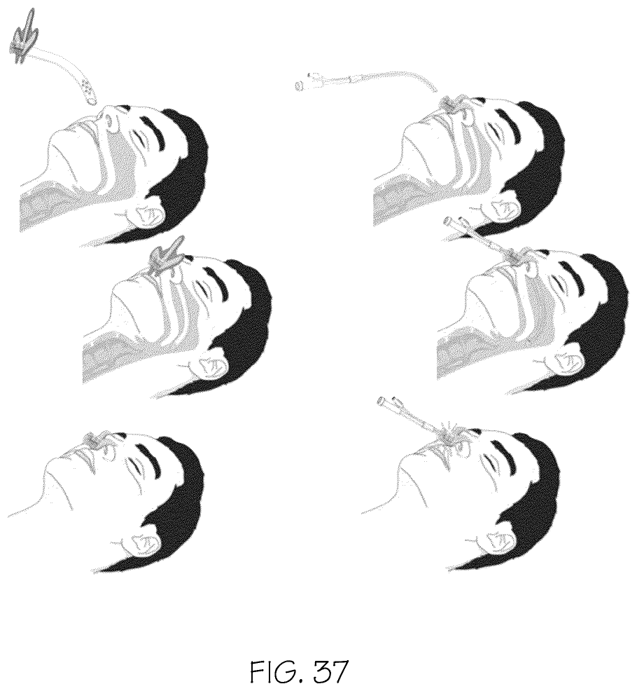

Operation of the nasal tube device 100 for securing a nasal tube 102 and one or more other tubes 122 to a nose of a patient is illustrated in FIG. 37. The nasal tube device 100 can be used according to the following steps. First, the nasal tube device 100 is placed in the patient with an adhesive liner 156 in place covering at least major portions of the one or more strip portions 136 of the adhesive tape 132 and with the clip 162 open. Then the adhesive liner 156 is removed and the adhesive tape 132 is secured to the face of the patient at the nose of the patient. Then the one or more other tubes 120 are introduced and advanced through the nasal tube lumen 118 of the nasal tube 102. For example, for a nasal tube 102 that comprises a nasopharyngeal airway for providing airway patency, the one or more other tubes 120 can include a tube for oxygen delivery, such as an oxygen delivery line, to provide retropalatal delivery of oxygen, and a tube for carbon dioxide monitoring, such as a capnography sampling line, for retropalatal sampling of exhaled breath for capnography monitoring. The position, including the depth, of the one or more other tubes 120 can be adjusted independently as needed. For example, a tube for oxygen delivery and a tube for carbon dioxide monitoring can be adjusted independently to achieve a satisfactory capnograph readout. Independent adjustment of such tubes is important for obtaining a quality capnograph readout and avoiding oxygen washout of the respiratory signal. Finally, the clip 162 is closed about the proximal-clip portion 106 of the nasal tube 102, thereby securing the nasal tube 102, the rest of the nasal tube device 100, and the one or more other tubes 120 in place on the patient. As will be appreciated, the order of these steps can be varied in various way while still achieving the same result.

As shown in FIGS. 1-10, a system 200 for securing a nasal tube 102 and one or more other tubes 120 to a nose of a patient also is disclosed. The system 200 comprises the nasal tube device 100 comprising (a) a nasal tube 102, (b) a flange 124, (c) an adhesive tape 132, and (d) a clip 162 as described above. The system 200 also comprises one or more other tubes 120. The one or more other tubes 120 are partially disposed in the nasal tube lumen 188 of the nasal tube 102 through the proximal opening 114 of the nasal tube 102. The clip 162 is reversibly closed about the proximal-clip portion 106 of the nasal tube 102, thereby reversibly compressing the proximal-clip portion 106 of the nasal tube 102 about the one or more other tubes 120.

In some embodiments, the nasal tube 102 comprises a nasopharyngeal airway, and the one or more other tubes 120 comprise a tube for oxygen delivery and a tube for carbon dioxide monitoring.

The system 200 can be used as described above for the nasal tube device 100.

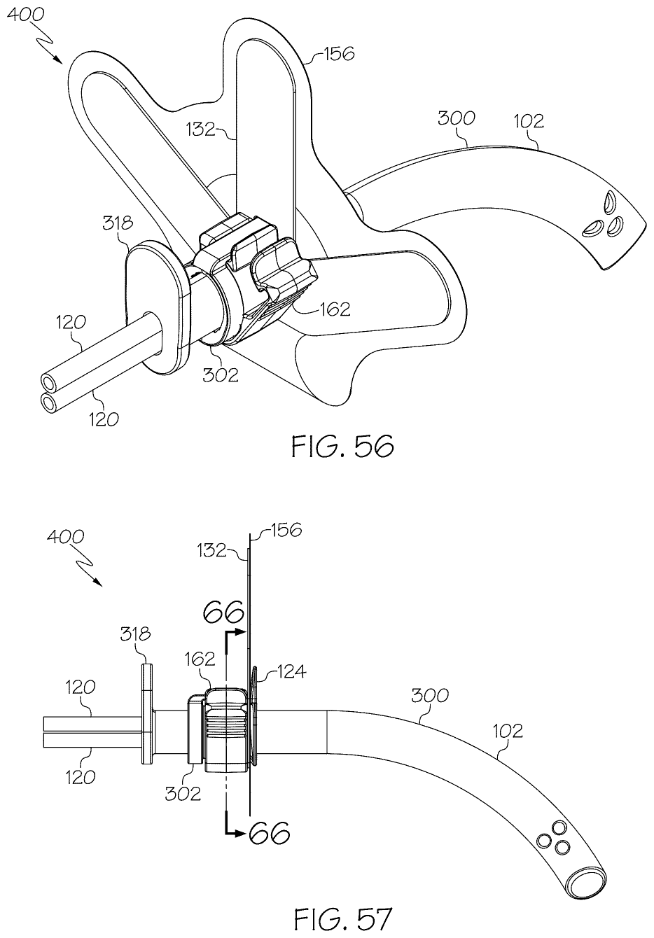

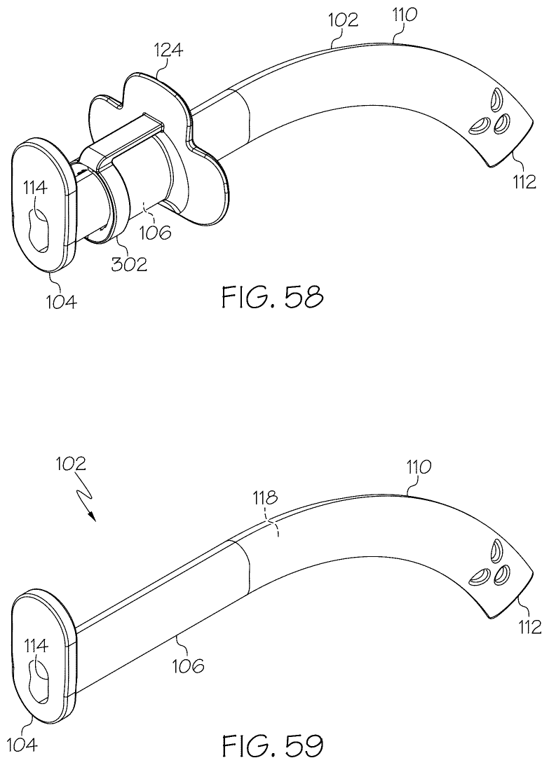

Turning to our nasal tube device comprising (a) a nasal tube, (b) a bolster, (c) a flange, (d) an adhesive tape, and (e) a clip, as described above, in more detail, FIGS. 56-57 show an embodiment of our nasal tube device 300 comprising (a) a nasal tube 102, (b) a bolster 302, (c) a flange 124, (d) an adhesive tape 132, and (c) a clip 162 that can be used for securing a nasal tube 102 and one or more other tubes 120 to a nose of a patient, as part of a system 400 comprising the nasal tube device 300 and the one or more other tubes 120. Our nasal tube device 300 is similar to our nasal tube device 100, except that our nasal tube device 300 further comprises a bolster 302 that is made separately from the nasal tube 102 and that can be slidable along the nasal tube 102. This allows for flexibility in positioning and reversible fixation of the bolster 302 and flange 124 along the nasal tube 102, and thus flexibility in positioning and securement of the nasal tube 102 to a patient in view of the specific anatomy of the patient.

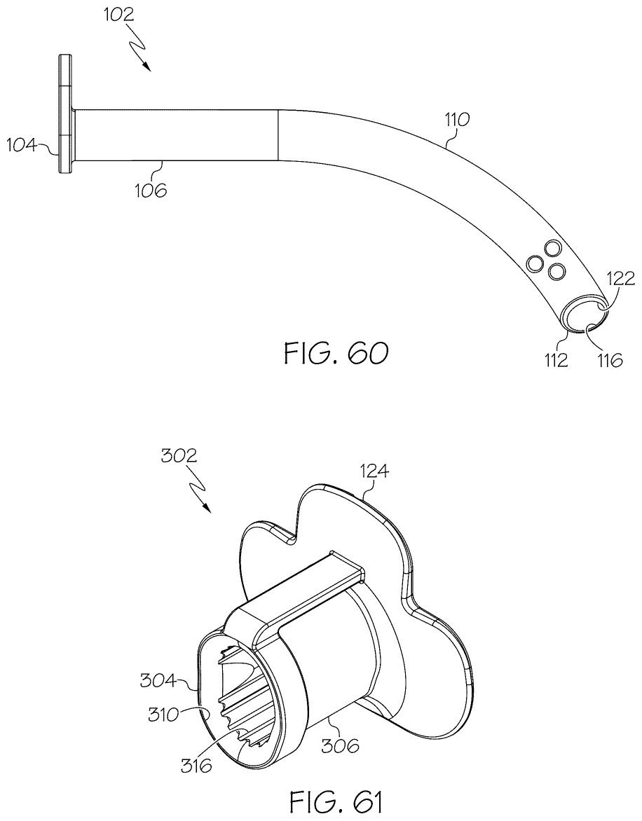

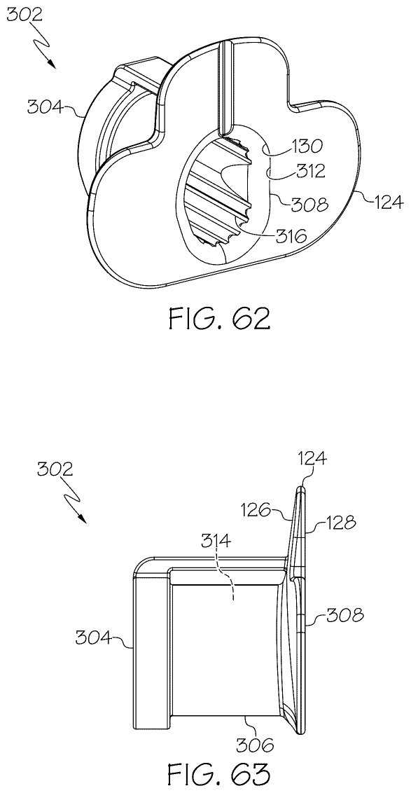

As shown in FIGS. 56-57, with reference to FIGS. 58-60, the nasal tube device 300 comprises a nasal tube 102.

The nasal tube 102 can be any kind of suitable nasal tube as described above, such as a nasopharyngeal airway, a nasogastric tube, or a nasojejunal tube, among other nasal tubes.

Accordingly, in some embodiments the nasal tube 102 comprises a nasopharyngeal airway, a nasogastric tube, or a nasojejunal tube.

As shown in FIGS. 58-60, the nasal tube 102 comprises, sequentially, a proximal end 104, a proximal-clip portion 106, a distal portion 110, and a distal end 112.

The proximal end 104 of the nasal tube 102 has a proximal opening 114, the distal end 112 of the nasal tube 102 has a distal opening 116, as described above, and the nasal tube 102 has a nasal tube lumen 118 extending from the proximal opening 114 of the nasal tube 102 to the distal opening 116 of the nasal tube 102, also as described above. The nasal tube lumen 118 can have an inner diameter sufficiently large to fit one or more other tubes 120, and can used for placement of the one or more other tubes 120, as described above.

The nasal tube 102 also has an elliptical tubular cross section 122 that is noncircular, as described above. Accordingly, the nasal tube 102 has a tubular cross section that has a generally ovoid shape rather than, for example, a generally circular shape. The nasal tube 102 having the elliptical tubular cross section 122 that is noncircular is advantageous as discussed above.

The nasal tube 102 may be made from, for example, one or more of silicone rubber elastomer, natural latex rubber, thermoplastic elastomer, styrene block copolymer, thermoplastic polyolefin, thermoplastic polyurethane, thermoplastic vulcanizate, ethylene-propylene-diene-monomer, copolymer derivatives of ethylene-propylene-diene-monomer, styrene-butadiene-rubber, copolymer derivatives of styrene-butadiene-rubber, flexible non-elastomeric polymer, or plasticized polyvinyl chloride, as discussed above.

A lubricant may be applied to one or more portions of surfaces of the nasal tube 102 to reduce friction during placement, also as noted above.

As shown in FIGS. 56-57, with reference to FIG. 58 and FIGS. 61-63, the nasal tube device 300 also comprises a bolster 302. The bolster 302 comprises a proximal end 304, a bolster body 306, and a distal end 308.

The proximal end 304 of the bolster 302 has a proximal opening 310, the distal end 308 of the bolster 302 has a distal opening 312, and the bolster 302 has a bolster lumen 314 extending from the proximal opening 310 of the bolster 302 to the distal opening 312 of the bolster 302.

The bolster 302 is formed separately from the nasal tube 102.

Like the nasal tube 102, the bolster 302 can be made from, for example, one or more of silicone rubber elastomer, natural latex rubber, thermoplastic elastomer, styrene block copolymer, thermoplastic polyolefin, thermoplastic polyurethane, thermoplastic vulcanizate, ethylene-propylene-diene-monomer, copolymer derivatives of ethylene-propylene-diene-monomer, styrene-butadiene-rubber, copolymer derivatives of styrene-butadiene-rubber, flexible non-elastomeric polymer, or plasticized polyvinyl chloride.

The nasal tube 102 extends through the bolster lumen 314. This can be accomplished for example by placing the nasal tube 102 into the bolster lumen 314 at the distal end 112 of the nasal tube 102 or the proximal end 104 of the nasal tube 102, such that the nasal tube 102 extends through the bolster lumen 314. The bolster 302 can then be slid along the nasal tube 102 to a position suitable for securement of the nasal tube 102 to the patient in view of the specific anatomy of the patient. This position thereby becomes the proximal-clip portion 106 of the nasal tube 102 of the nasal tube device 300.

As shown in FIGS. 56-57, with reference to FIG. 58 and FIGS. 61-63, the nasal tube device 300 also comprises a flange 124. The flange 124 has a proximal face 126 and a distal face 128, as described above. The flange 124 extends radially outwardly from the nasal tube 102, also as described above. The flange 124 has a hole 130 between the proximal face 126 of the flange 124 and the distal face 128 of the flange 124, as described above. The flange 124 is formed separately from the nasal tube 102. The nasal tube 102 extends through the hole 130 of the flange 124. The flange 124 is distal to the bolster 302 on the nasal tube 102.

The flange 124 is highly compliant based on being thin and flexible and provides a barrier to moisture and heat based on its composition, as described above. Suitable thicknesses for the flange 124, suitable tensile strengths, tensile moduli, and hardnesses of the material from which the flange 124 is made, and suitable materials for making the flange 124 are as described above. Making a flange 124 that is highly compliant based on being thin and flexible and that provides a barrier to moisture and heat based on its composition is important to the performance of the bond between the adhesive tape 132 and the flange 124 as described above.

Accordingly, in some embodiments the flange 124 has a thickness of 0.50 to 5.00 mm, 0.60 to 2.50 mm, or 0.75 to 1.00 mm.

Also in some embodiments the flange 124 is made from a material having (i) a tensile strength of 2.0 to 20 MPa, a tensile modulus of 0.5 to 10 MPa, and a hardness of 50 Shore-00 to 90 Shore A, (ii) a tensile strength of 5 to 15 MPa, a tensile modulus of 1 to 5 MPa, and a hardness of 30 to 70 Shore A, or (iii) a tensile strength of 8 to 10 MPa, a tensile modulus of 2 to 3 MPa, and a hardness of 40 to 60 Shore A.

Also in some embodiments the flange 124 is made from one or more of silicone rubber elastomer, natural latex rubber, thermoplastic elastomer, styrene block copolymer, thermoplastic polyolefin, thermoplastic polyurethane, thermoplastic vulcanizate, ethylene-propylene-diene-monomer, copolymer derivatives of ethylene-propylene-diene-monomer, styrene-butadiene-rubber, copolymer derivatives of styrene-butadiene-rubber, flexible non-elastomeric polymer, or plasticized polyvinyl chloride.

The flange 124 can be made to extend from the bolster 302 at the distal end 308 of the bolster 302. This can be accomplished, for example, by making the flange 124 integral to the bolster 302, or by forming the flange 124 separately from the bolster 302 and then attaching the flange 124 to the bolster 302. In either case and other cases, having the flange 124 extend from the bolster 302 can be useful for providing sufficient support for the flange 124 so that it can extend radially outwardly from the nasal tube 102. This also can allow placement of the flange 124 on the nasal tube 102 simultaneously with placement of the bolster 302 on the nasal tube 102.

Accordingly, in some embodiments, the flange 124 extends from the bolster 302 at the distal end 308 of the bolster 302. In some of these embodiments the flange 124 is integral to the bolster 302. In some of these embodiments, the flange 124 is formed separately from the bolster 302 and is attached to the bolster 302.

As shown in FIGS. 56-57, the nasal tube device 300 also comprises an adhesive tape 132, as described above. Thus, the adhesive tape 132 comprises a base portion 134 and one or more strip portions 136 extending from the base portion 134, as described above. The base portion 134 of the adhesive tape 132 has a proximal side 138, a distal side 140, and a hole 142 therethrough, also as described above. Each of the one or more strip portions 136 has a proximal side 144 and a distal side 146, also as described above. The distal side 146 of each of the one or more strip portions 136 each has an adhesive surface 148, also as described above. The nasal tube 102 extends through the hole 142 of the base portion 134 of the adhesive tape 132, also as described above. The adhesive tape 132 is attached to the flange 124 at the base portion 134 of the adhesive tape 132, also as described above.

In some embodiments, the one or more strip portions 136 of the adhesive tape 132 comprise two to four strip portions 136, or three strip portions 136, extending from the base portion 134 of the adhesive tape 132.

In some embodiments, the adhesive tape 132 is attached to the flange 124 based on the distal side 140 of base portion 134 of the adhesive tape 132 being adhered to the proximal face 126 of the flange 124, as described above.

In some embodiments, the adhesive tape 132 is attached to the flange 124 based on the proximal side 138 of base portion 134 of the adhesive tape 132 being adhered to the distal face 128 of the flange 124, as described above.

As shown in FIGS. 56-57, in some embodiments, the nasal tube device 300 further comprises an adhesive liner 156, the adhesive liner 156 covering the adhesive surfaces 148 of the distal sides 146 of the one or more strip portions 136 of the adhesive tape 132, as described above.

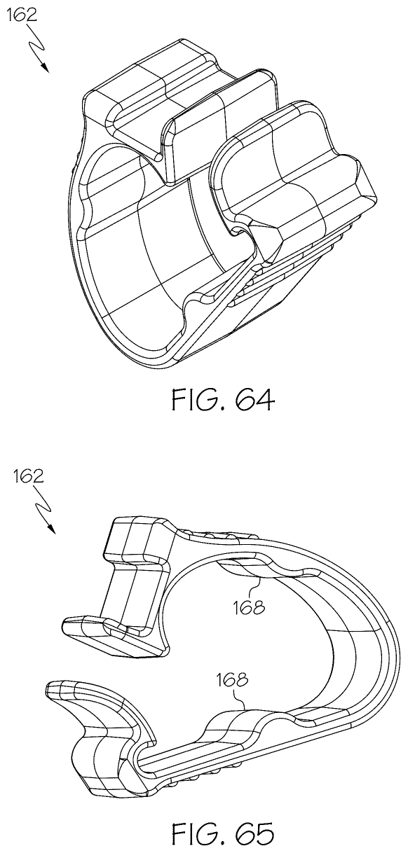

As shown in FIGS. 56-57, with reference to FIGS. 64-65, the nasal tube device 300 also comprises a clip 162 for securing one or more other tubes 120 to the nasal tube 102 within the nasal tube lumen 118 of the nasal tube 102 at the proximal-clip portion 106 of the nasal tube 102. The clip 162 is reversibly closeable about the bolster 302 to reversibly compress the proximal-clip portion 106 of the nasal tube 102 about the one or more other tubes 120 when closed.

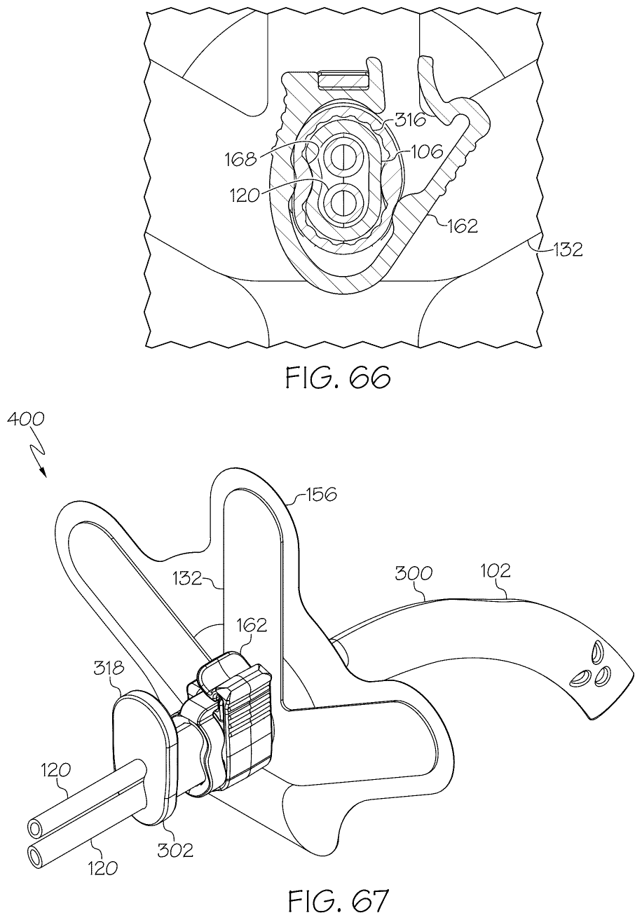

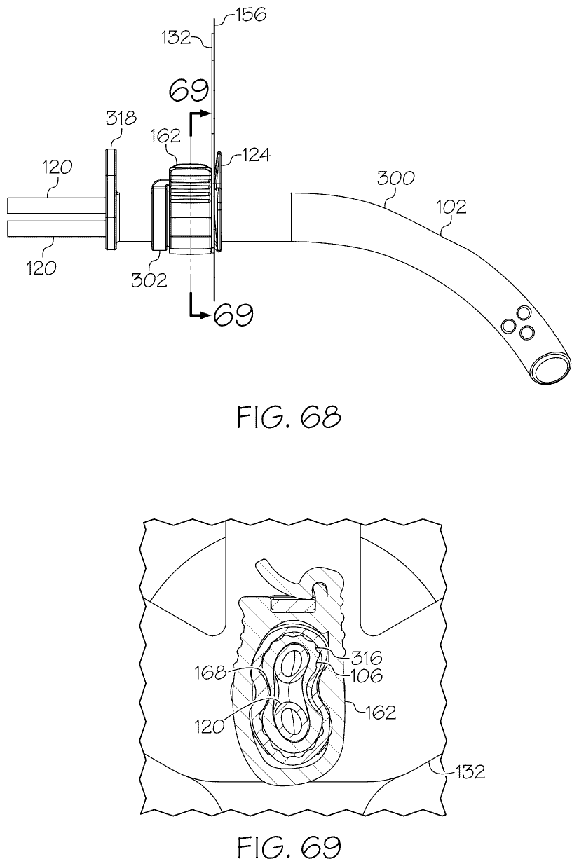

As shown in FIGS. 56-57, FIGS. 61-62, and FIGS. 64-69, one or more of the clip 162 and the bolster 302 can be made with one or more protrusions 168, 316 to reversibly compress the proximal-clip portion 106 of the nasal tube 102 about the one or more other tubes 120 when the clip 162 is closed about the proximal-clip portion 106 of the nasal tube 102. This can promote holding of the one or more other tubes 120 in place within the nasal tube lumen 118 of the nasal tube 102 when the clip 162 is closed, and thus prevention of displacement of the one or more other tubes 120.

Accordingly, in some embodiments one or more of the clip 162 or the bolster 302 comprise one or more protrusions 316 to reversibly compress the proximal-clip portion 106 of the nasal tube 102 about the one or more other tubes 120 when the clip 162 is closed about the bolster 302.

Optionally, the clip 162 can be attached to the bolster 302, such that the clip 162 remains attached to the bolster 302 even when the clip 162 is open. For example, a portion of the clip 162 can be adhered to a portion of the bolster 302. Similarly as for the clip 162 and the proximal-clip portion 106 of the nasal tube 102 of the nasal tube device 100, this can be useful for preventing an end user from inadvertently dropping the clip 162 during handling and for preventing a patient from inadvertently swallowing the clip 162 if dropped.

Accordingly, in some embodiments the clip 162 is adhered to the bolster 302.

The clip 162 can be made, for example, for polypropylene, polyethylene, polystyrene, acrylonitrile-styrene, cyclic-olefin-copolymer, polyester-terephthalate, polycyclohexylenedimethylene-terepthalate, copolyester, acrylonitrile-butadiene-styrene, polyacetal, polycarbonate, polyvinylchloride, polylactic-acid, polyimide, polyamide, polyetherimide, polysulfone, polyethersulfone, liquid crystal polymer, polymethyl-methacrylate, polyphthalamide, polyphenylene-oxide, polyphenylene-sulfide, polyphenylene sulfone, syndiotactic-polystyrene, polyaryletherketone, and polyetheretherketone, as discussed above.

The bolster 302 of the nasal tube device 300 can be slidable along the nasal tube 102 when the clip 162 is not reversibly closed about the bolster 302, and the flange 124 also can be slidable along the nasal tube 102. The nasal tube 102 also can further comprise a rim 318 extending radially outwardly from the nasal tube 102 at the proximal end 104 of the nasal tube 102, the rim 318 having an outer diameter greater than an inner diameter of the bolster lumen 314 and an inner diameter of the hole 142 of the base portion 134 of the adhesive tape 132. For a nasal tube device 300 in which the bolster 302 and the flange 124 can be so slidable, having the rim 318 having an outer diameter greater than an inner diameter of the bolster lumen 314 and an inner diameter of the hole 142 of the base portion 134 of the adhesive tape 132 can be useful for preventing the bolster 302, the flange 124, and the adhesive tape 132 from sliding off of the nasal tube 102 at the proximal end 104 of the nasal tube 102 while the clip 162 remains open, prior to securing the nasal tube 102 in place on a patient.

Accordingly, in some embodiments the bolster 302 is slidable along the nasal tube 102 when the clip 162 is not reversibly closed about the bolster 302, and the flange 124 is slidable along the nasal tube 102. In some of these embodiments, the nasal tube 102 further comprises a rim 318 extending radially outwardly from the nasal tube 102 at the proximal end 104 of the nasal tube 102, the rim 318 having an outer diameter greater than an inner diameter of the bolster lumen 314 and an inner diameter of the hole 142 of the base portion 134 of the adhesive tape 132.

The nasal tube device 300 can be used according to the following steps. First, the bolster 302 and flange 124 are placed on the nasal tube 102 of the nasal tube device 300 and are slid therealong to a position suitable for securement of the nasal tube 102 to a patient in view of the specific anatomy of the patient, this position becoming the proximal-clip portion 106 of the nasal tube 102. Then the nasal tube device 300 is placed in the patient with an adhesive liner 156 in place covering at least major portions of the one or more strip portions 136 of the adhesive tape 132 and with the clip 162 open. Then the adhesive liner 156 is removed and the adhesive tape 132 is secured to the face of the patient at the nose of the patient. Then one or more other tubes 120 are introduced and advanced through the nasal tube lumen 118 of the nasal tube 102. The position, including the depth, of the one or more other tubes 120 can be adjusted independently as needed. Finally, the clip 162 is closed about the bolster 302, thereby securing the nasal tube 102, the rest of the nasal tube device 300, and the one or more other tubes 120 in place on the patient. The order of these steps also can be varied in various ways while still achieving the same result.

As shown in FIGS. 56-57, another system 400 for securing a nasal tube 102 and one or more other tubes 120 to a nose of a patient also is disclosed. The system 400 comprises the nasal tube device 300 comprising (a) a nasal tube 102, (b) a bolster 302, (c) a flange 124, (d) an adhesive tape 132, and (c) a clip 162, as described above. The system 400 also comprises one or more other tubes 120. The one or more other tubes 120 are partially disposed in the nasal tube lumen 118 of the nasal tube 102 through the proximal opening 114 of the nasal tube 102. The clip 162 is reversibly closed about the bolster 302, thereby reversibly compressing the proximal-clip portion 106 of the nasal tube 102 about the one or more other tubes 120.

In some embodiments, the nasal tube 102 comprises a nasopharyngeal airway, and the one or more other tubes 120 comprise a tube for oxygen delivery and a tube for carbon dioxide monitoring.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention.

What is claimed is:

1. A nasal tube device for securing a nasal tube and one or more other tubes to a nose of a patient, the nasal tube device comprising:
(a) a nasal tube comprising, sequentially, a proximal end, a proximal-clip portion, a proximal-flange portion, a distal portion, and a distal end, the proximal end of the nasal tube having a proximal opening, the distal end of the nasal tube having a distal opening, and the nasal tube having a nasal tube lumen extending from the proximal opening of the nasal tube to the distal opening of the nasal tube;
(b) a flange having a proximal face and a distal face, the flange extending radially outwardly from the proximal-flange portion of the nasal tube, the nasal tube extending through the flange;
(c) an adhesive tape comprising a base portion and one or more strip portions extending from the base portion, the base portion of the adhesive tape having a proximal side, a distal side, and a hole therethrough, each of the one or more strip portions having a proximal side and a distal side, the distal side of each of the one or more strip portions each having an adhesive surface, the nasal tube extending through the hole of the base portion of the adhesive tape, the adhesive tape being attached to the flange at the base portion of the adhesive tape; and
(d) a clip for securing one or more other tubes to the nasal tube within the lumen of the nasal tube at the proximal-clip portion of the nasal tube, the clip being reversibly closeable about the proximal-clip portion of the nasal tube to reversibly compress the proximal-clip portion of the nasal tube about the one or more other tubes when closed, wherein:
the flange is highly compliant based on being thin and flexible and provides a barrier to moisture and heat based on its composition; and
the nasal tube has an elliptical tubular cross section that is noncircular.

2. The nasal tube device according to claim 1, wherein the nasal tube comprises a nasopharyngeal airway, a nasotracheal tubes, a nasogastric tube, or a nasojejunal tube.

3. The nasal tube device according to claim 1, wherein the flange is formed integrally to the nasal tube at the proximal-flange portion of the nasal tube.

4. The nasal tube device according to claim 1, wherein the flange is formed separately from the nasal tube, the flange has a hole therethrough, and the nasal tube extends through the hole of the flange.

5. The nasal tube device according to claim 1, wherein the flange has a thickness of 0.50 to 5.00 mm.

6. The nasal tube device according to claim 1, wherein the flange is made from a material having a tensile strength of 2.0 to 20 MPa, a tensile modulus of 0.5 to 10 MPa, and a hardness of 50 Shore-00 to 90 Shore A.

7. The nasal tube device according to claim 1, wherein the flange is made from one or more of silicone rubber elastomer, natural latex rubber, thermoplastic elastomer, styrene block copolymer, thermoplastic polyolefin, thermoplastic polyurethane, thermoplastic vulcanizate, ethylene-propylene-diene-monomer, copolymer derivatives of ethylene-propylene-diene-monomer, styrene-butadiene-rubber, copolymer derivatives of styrene-butadiene-rubber, flexible non-elastomeric polymer, or plasticized polyvinyl chloride.

8. The nasal tube device according to claim 1, wherein the one or more strip portions of the adhesive tape comprise two to four strip portions extending from the base portion of the adhesive tape.

9. The nasal tube device according to claim 1, wherein the adhesive tape is attached to the flange based on the distal side of base portion of the adhesive tape being adhered to the proximal face of the flange.

10. The nasal tube device according to claim 1, wherein the adhesive tape is attached to the flange based on the proximal side of base portion of the adhesive tape being adhered to the distal face of the flange.

11. The nasal tube device according to claim 1, wherein the nasal tube device further comprises an adhesive liner, the adhesive liner covering the adhesive surfaces of the distal sides of the one or more strip portions of the adhesive tape.

12. The nasal tube device according to claim 1, wherein the clip and the proximal-clip portion of the nasal tube comprise structures that provide a complementary fit between the clip and the proximal-clip portion of the nasal tube when the clip is closed about the proximal-clip portion of the nasal tube.

13. The nasal tube device according to claim 1, wherein one or more of the clip or the proximal-clip portion of the nasal tube comprise one or more protrusions to reversibly compress the proximal-clip portion of the nasal tube about the one or more other tubes when the clip is closed about the proximal-clip portion of the nasal tube.

14. The nasal tube device according to claim 1, wherein the nasal tube has an elliptical tubular cross section that is noncircular based on the elliptical tubular cross section having a major axis length and an interfocal length, and the interfocal length being 0.50 to 0.99 times the major axis length.

15. A system for securing a nasal tube and one or more other tubes to a nose of a patient, the system comprising the nasal tube device according to claim 1 and one or more other tubes, wherein:
the one or more other tubes are partially disposed in the nasal tube lumen of the nasal tube through the proximal opening of the nasal tube; and
the clip is reversibly closed about the proximal-clip portion of the nasal tube, thereby reversibly compressing the proximal-clip portion of the nasal tube about the one or more other tubes.

16. The system according to claim 15, wherein the nasal tube comprises a nasopharyngeal airway, and the one or more other tubes comprise a tube for oxygen delivery and a tube for carbon dioxide monitoring.

17. A nasal tube device for securing a nasal tube and one or more other tubes to a nose of a patient, the nasal tube device comprising:
(a) a nasal tube comprising, sequentially, a proximal end, a proximal-clip portion, a distal portion, and a distal end, the proximal end of the nasal tube having a proximal opening, the distal end of the nasal tube having a distal opening, and the nasal tube having a nasal tube lumen extending from the proximal opening of the nasal tube to the distal opening of the nasal tube;
(b) a bolster comprising a proximal end, a bolster body, and a distal end, the proximal end of the bolster having a proximal opening, the distal end of the bolster having a distal opening, the bolster having a bolster lumen extending from the proximal opening of the bolster to the distal opening of the bolster, the bolster being formed separately from the nasal tube, the nasal tube extending through the bolster lumen;
(c) a flange having a proximal face and a distal face, the flange extending radially outwardly from the nasal tube, the flange having a hole between the proximal face of the flange and the distal face of the flange, the flange being formed separately from the nasal tube, the nasal tube extending through the hole of the flange, the flange being distal to the bolster on the nasal tube;
(d) an adhesive tape comprising a base portion and one or more strip portions extending from the base portion, the base portion of the adhesive tape having a proximal side, a distal side, and a hole therethrough, each of the one or more strip portions having a proximal side and a distal side, the distal side of each of the one or more strip portions each having an adhesive surface, the nasal tube extending through the hole of the base portion of the adhesive tape, the adhesive tape being attached to the flange at the base portion of the adhesive tape; and
(e) a clip for securing one or more other tubes to the nasal tube within the lumen of the nasal tube at the proximal-clip portion of the nasal tube, the clip being reversibly closeable about the bolster to reversibly compress the proximal-clip portion of the nasal tube about the one or more other tubes when closed, wherein:
the flange is highly compliant based on being thin and flexible and provides a barrier to moisture and heat based on its composition; and
the nasal tube has an elliptical tubular cross section that is noncircular.

18. The nasal tube device according to claim 17, wherein the flange extends from the bolster at the distal end of the bolster.

19. The nasal tube device according to claim 17, wherein the bolster is slidable along the nasal tube when the clip is not reversibly closed about the bolster, and the flange is slidable along the nasal tube.

20. A system for securing a nasal tube and one or more other tubes to a nose of a patient, the system comprising the nasal tube device according to claim 17 and one or more other tubes, wherein:
the one or more other tubes are partially disposed in the nasal tube lumen of the nasal tube through the proximal opening of the nasal tube; and
the clip is reversibly closed about the bolster, thereby reversibly compressing the proximal-clip portion of the nasal tube about the one or more other tubes.
